(19) United States
(12) Patent Application Publication
(10) Pub. No.: US 2012/0156674 A1
(43) Pub. Date: Jun. 21, 2012

(54) METHODS OF DIFFERENTIATING BETWEEN NON-GENOTOXIN AND GENOTOXIN-ASSOCIATED TUMORS

(76) Inventors: Sydney D. FINKELSTEIN, Pittsburgh, PA (US); Eric Matthew Gayle Ellsworth, Pittsburgh, PA (US)

(21) Appl. No.: 13/331,966

(22) Filed: Dec. 20, 2011

Related U.S. Application Data
(60) Provisional application No. 61/425,109, filed on Dec. 20, 2010.
(60) Provisional application No. 61/443,014, filed on Feb. 15, 2011.

(51) Int. Cl. C12Q 1/68

(52) U.S. Cl. 435/6.11

(57) ABSTRACT

Embodiments of the present disclosure are directed to methods of differentiation of non-genotoxin associated versus genotoxin-associated tumors.

### CROSS REFERENCE

This application claims the benefit of U.S. Provisional Application Ser. No. 61/425,109 entitled “Molecular Discrimination Between Sporadic Versus Toxin-Associated Cancer Formation” filed Dec. 20, 2010 and U.S. Provisional Application Ser. No. 61/443,014 entitled “Methods of Mutational Profiling of Non-Genotoxin Versus Genotoxin-Associated Tumors”, filed on Feb. 15, 2011; each of which is incorporated herein by reference in their entireties.

### GOVERNMENT INTERESTS

Not Applicable

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

Not Applicable

### BRIEF SUMMARY OF THE INVENTION

Embodiments of the present disclosure are directed to methods of differentiating non-genotoxin associated tumors from a genotoxin-associated tumor comprising ; and counting the number of loci in a set of loci spanning a segment of a genome containing no tumor suppressor genes with copy number imbalance. In one embodiment, a tumor is determined to be genotoxin-associated when about 20% or more of the measured loci show copy number imbalance. In some embodiments, the method may further comprise measuring copy number imbalance at the loci. In embodiments, the method may further comprise selected a set of loci spanning a segment of a genome containing no tumor suppressor genes.

In another embodiment, a method of differentiating a non-genotoxin associated from a genotoxin-associated tumor may further comprise determining the presence of a point mutation in a VHL gene, wherein a point mutation in distal portion of exon 1 is indicative of a genotoxin-associated tumor. In yet another embodiment, the distal portion of exon 1 may comprises codon 81 of the VHL gene.

In another embodiment, a method of differentiating a non-genotoxin associated from a genotoxin-associated tumor may further comprise determining the presence of a point mutation in exons 1, 2 and 3 of the VHL gene, wherein the presence of more than one point mutation in the exons is indicative of a genotoxin-associated tumor.

In another embodiment, a genotoxin may comprise a carcinogen, a chlorinated hydrocarbon, a polycyclic aromatic hydrocarbon, a benzene, aflatoxin, or a combination thereof.

In some embodiments, the chlorinated hydrocarbon comprises trichloroethylene, vinyl chloride or a combination thereof.

In one embodiment, a non-genotoxin associated tumor may comprise a sporadic tumor. In yet another embodiment, a genotoxin-associated tumor may be from a subject who has a cancer. In another embodiment, the cancer may be selected from colon, brain, breast, kidney, leukemia, prostate, uterus, stomach, lymphoma, esophagus, sarcoma, thyroid, hemangioblastoma and combinations thereof.

In yet another embodiment the method may further comprise determining passenger fraction allelic loss mutation rate in a segment of a genome, wherein fraction allelic loss mutation rate of about 20% or more is indicative of a toxin associated tumor. In some embodiments, the segment of a genome comprises chromosome 3, chromosome 4, chromosome 12, chromosome 16 or a combination thereof. In yet other embodiments, the segment of a genome does not contain a tumor suppressor gene.

### BRIEF DESCRIPTION OF THE DRAWINGS

For a fuller understanding of the nature and advantages of the present invention, reference should be made to the following detailed description taken in connection with the accompanying drawings, in which:

FIG. 1 illustrates the migration of the plume to the west that caused contamination of potable water supplies of many residents of Shannon. Extremely and persistently high concentrations of trichloroethylene were found within a particular area of Shannon; this area has been termed the “Red Zone”.

FIG. 2 Illustrates chromosome 3 ideogram showing the approximate location of the microsatellite markers used in the survey of passenger mutational change. The position of the markers was based on database information (www.ensembl.org).

FIG. 3 Illustrates Passenger mutational change model. During an event of clonal expansion, both critical growth regulatory gene driver mutations as well as co-existing passenger mutational change will be fixed into the most actively proliferating tumor cell population. In sporadic cancer, the pace of mutational damage is relatively slow and the opportunity for DNA repair will be greater resulting is a lower cumulative amount of mutational damage being present at the step of clonal expansion. In contrast, when DNA damage is intense and/or when mechanisms for repair DNA damage are reduced, the load of accumulated mutational change will be greater in the neoplastic cell population undergoing clonal expansion.

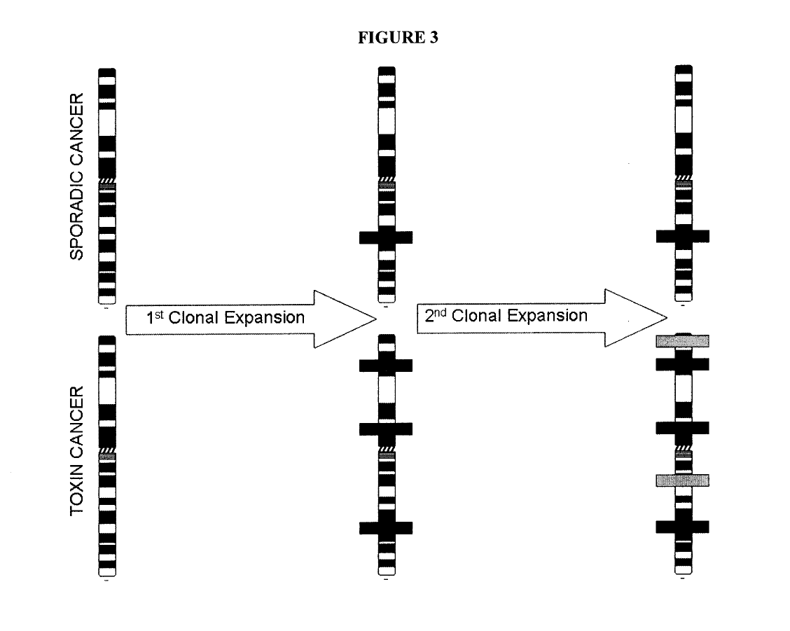

FIG. 4 Illustrates DNA sequencing electropherogram of hemangioblastoma arising in chlorinated solvent exposed subject. Point mutation involves codon 89 in microdissected tumor tissue. The non-neoplastic tissue did not show this alteration.

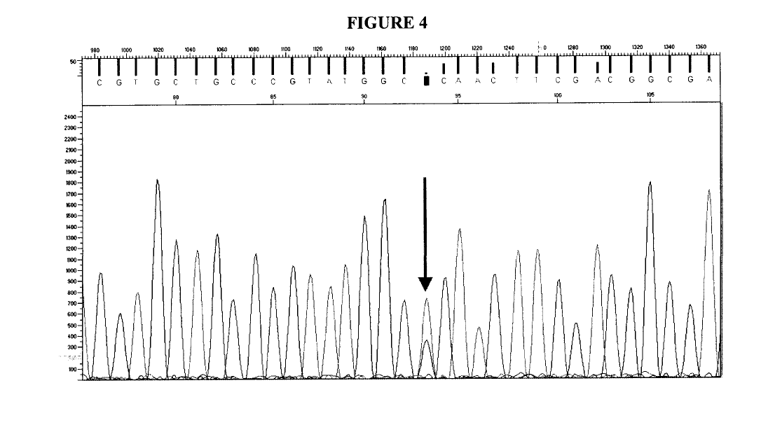

FIG. 5; FIG. 5a illustrates a cutoff of FAL≧0.26 to predict toxin exposure versus sporadic cancer formation. Applying this cutoff to the cohort of Example 2 correctly predicts exposure status for all but one false negative exposed case, yielding a sensitivity of 96% [95% CI: 80-100%], a specificity of 100% [95% CI: 69-100%], and an overall accuracy of 97% [95% CI: 85-100%]. FIG. 5b: For absolute numbers of mutations, the previous cutoff of≧3 mutations was evaluated, yielding slightly lower accuracy of 92% [95% CI: 78-98%].

### DETAILED DESCRIPTION

This invention is not limited to the particular processes, compositions, or methodologies described, as these may vary. The terminology used in the description is for the purpose of describing the particular versions or embodiments only, and it is not intended to limit the scope of the present invention. All publications mentioned herein are incorporated by reference in their entirety. Nothing herein is to be construed as an admission that the invention is not entitled to antedate such disclosure by virtue of prior invention.

As used herein, the singular forms “a,” “an,” and “the” include plural references unless the context clearly dictates otherwise. In addition, the word “comprising” as used herein is intended to mean “including but not limited to.” Unless defined otherwise, all technical and scientific terms used herein have the same meanings as commonly understood by one of ordinary skill in the art.

As used herein, all claimed numeric terms are to be read as being preceded by the term “about,” which means plus or minus 10% of the numerical value of the number with which it is being used. Therefore, a claim to “50%” means “about 50%” and encompasses the range of 45%-55%.

As used herein, a “genotoxin” may comprise a genotoxic compound, an environmental factor and combinations thereof. Genotoxic compounds include, but are not limited to, a chlorinated hydrocarbon, aflatoxin, trichloroethylene, tobacco smoke (tobacco use or as second hand smoke), arsenic, asbestos, crystalline silica, benzenes, penzo[a]pyrene, beryllium, bis(chloro)methyl ether, 1,3-butadiene, chromium V1 compounds, coal tar, pitch, nickel compounds, soots, mustard gas, erionite, nickel compounds, heterocyclic amines, vinyl chloride, thorium dioxide, phenacetin, 4-aminobiophenyl, benzidine, 2-naphthylamine, phenacetin, cadmium, cyclosporine A, ethylene oxide, N-nitroso compounds, nitric oxide, antineoplastic agents, chemotherapeutic agents, compounds that cause free oxygen radicals and combinations thereof, Environmental factors include, but are not limited to, viruses, such as certain RNA viruses (e.g., retroviruses such as human T-lymphotropic virus type 1 and type 2, human immunodeficiency virus, and hepatitis C virus) and DNA viruses (such as hepadnaviruses, papillomaviruses, EpsteinBarr virus, Kaposi's sarcoma-associated herpesvirus, simian vacuolating virus 40 and other polyomaviruses), sun exposure, radiation and the like. In some embodiments, a genotoxin is a known carcinogen. In some embodiments a carcinogen is meant to include a substance either known or reasonably anticipated to cause cancer in humans in certain situations. In some embodiments, cancer may develop only after prolonged exposure. In some embodiments, with certain substances or exposure circumstances, cancer may develop after even brief exposure. The carcinogenic nature of any carcinogen depends on many factors including but not limited to the intrinsic carcinogenicity of the substance, the amount and duration of exposure, and the individual's susceptibility to the carcinogenic action of the substance.

As used herein, a “genotoxin-associated tumor” refers to a tumor that is caused by exposure to a genotoxin or a tumor that is progressing, growing, developing or increasing (such as in size and/or mass and/or location) because of exposure to a genotoxin. “Genetic acquisition due to environmental factor” is also used to refer to a genotoxin associated tumor. By “spontaneous mutation” is meant a mutation that occurs during the development of a cancer or a growth that is progressing towards cancer that is not a germ-line mutation. Spontaneous mutations are included within genotoxin associated tumors. In some embodiments, a genotoxin associated tumor refers to a tumor in a patient with known or demonstrable exposure to a genotoxin. In some embodiments, a genotoxin associated tumor refers to a tumor in a patient with likely or likely demonstrable exposure to a genotoxin. In some embodiments, a genotoxin-associated tumor that may have been caused by, or has been advanced by, genotoxin exposure.

As used herein, a “non-genotoxin-associated tumor” refers to a tumor that (i) is not caused by exposure to a genotoxin compound; (ii) is not progressing, growing, developing or increasing (such as in size and/or mass and/or location) because of exposure to a genotoxin; and (iii) is caused by a genomic deletion that is part of the subject's germ line. Such mutations occur in all or substantially all the cells and are not caused by a genotoxin nor do they arise spontaneously. “Genetic acquisition due to familial inheritance” is also used to refer to a non-genotoxin associated tumor. In some embodiments a non-genotoxin-associated tumor may be referred to as a sporadic tumor and may be defined as cancer occurring randomly in people with little or unknown family history of the disease and without known exposure to chlorinated solvents or other known genotoxic agents.

By “clonal expansion” is meant a unidirectional process replacing precursor neoplastic cells with a dominant tumor cell population of cells with progressively more mutations.

As used herein, mutations which result in clonal expansion are referred to as “driver” mutations. Drive mutations are generally directly involved in cancer development and progression. Clonal expansion occurs in response to DNA damage to specific oncogenes and tumor suppressor genes normally responsible for growth regulation.

As used herein, “passenger” mutations refer to mutations that are present in regions of the genome which do not harbor oncogenes or tumor suppressor genes which are also found to be frequently mutated in cancer. These mutations are called “passengers” since they do not necessarily drive further neoplastic progression. As a tumor cell population clonally expands, the tumor cells will carry not only driver mutations affecting critical growth regulatory genes but also co-existing passenger mutations that are present in the genome of the affected cell at times of clonal expansion.

“Tumor” is meant to include any malignant or non-malignant tissue or cellular containing material or cells. “Non-malignant tissue” is meant to include any abnormal tissue or cell phenotype and/or genotype associated with metaplasia, hyperplasia, a polyp, or pre-cancerous conditions (e.g., leukoplakia, colon polyps), regenerative change, physiologic adaption to stress or injury and cellular change in response to stress of injury. Tumor is also meant to include solid tumors as well as leukemias and lymphomas. “Neoplasm”, “malignancy”, and “cancer” are used interchangeably. “Normal tissue” refers to tissues of cellular phenotypes not associated with a tumor, metaplasia, hyperplasia, a polyp, or pre-cancerous conditions (e.g., leukoplakia, colon polyps), regenerative change, physiologic adaption to stress or injury and cellular change in response to stress of injury

As used herein, “loss of heterozygosity” is meant to include the loss of normal function of one allele of a gene in which the other allele was already inactivated. A common occurrence in cancer, loss of heterzygozity may indicate the absence of a functional tumor suppressor gene in a particular gene.

Embodiments of the present disclosure are directed to methods of differentiating a non-genotoxin associated tumors from a genotoxin-associated tumors comprising counting the number of loci spanning a segment of a genome not containing a tumor suppressor gene with copy number imbalance, wherein a tumor is a genotoxin-associated tumor when about 20% or more of the loci show copy number imbalance. In some embodiments, where about 20% or more of the loci show copy number imbalance, there has been intense genotoxin exposure. In some embodiments, where about 20% or more of the loci show copy number imbalance, a tumor may have been caused by, or advanced by, genotoxin exposure.

Embodiments of the present invention are further directed to methods of differentiating sporadic cancer formation and genotoxin-associated cancer formation. The method, applicable to all forms of cancer and suitable for use on virtually all archival fixative treated clinical specimens of cancer, quantitatively determines the amount of ‘passenger’ mutational damage over a defined region of the genome for loss of heterozygosity mutational change. In some embodiments, the test is based on the notion of “passenger” mutations in cancer and the “shotgun” nature of damage to the genome by trichloroethylene and similar chlorinated solvents.

Embodiments of the present invention are directed to methods of differentiating a non-genotoxin associated tumor from a genotoxin-associated tumors comprising counting the number of loci spanning a segment of a genome not containing a tumor suppressor gene with copy number imbalance, wherein a tumor is a genotoxin-associated tumor when about 20% or more of the loci show copy number imbalance.

In yet another embodiment, the method further comprises determining the presence of point mutations in the VHL gene, wherein a point mutation at nucleotide 484 is indicative of a tumor whose malignant progression has been advanced by genotoxic exposure. In yet another embodiment, the method further comprises determining the presence of point mutation in exons 1, 2 and 3 of the VHL gene, wherein the presence of more than one point mutation in exons 1, 2, 3 and combinations of point mutations is indicative of a tumor whose malignant progression has been advanced by genotoxic exposure.

In some embodiments, a genotoxin comprises a carcinogen. The U.S. Department of Health and Human Services Public Health Service National Toxicology Program provides a periodic report of known or reasonable anticipated to cause cancer in human beings in certain situation. The 2011 Report on Carcinogens provides a list of carcinogens comporting to the above definition. This report is hereby incorporated by reference in its entirety.

In another embodiment, a genotoxin may comprise a carcinogen, a chlorinated hydrocarbon, a polycyclic aromatic hydrocarbon, a benzene, aflatoxin, or a combination thereof.

In some embodiments, a chlorinated hydrocarbon comprises trichloroethylene, vinyl chloride or a combination thereof.

While some cancers can be attributed to known etiologic factors, in many patients the specific causation for cancer is unclear. Various cancer-initiating factors have been well-characterized, including oncogenic viruses (human papillomavirus associated with cervical squamous cell carcinoma, hepatitis B virus linked to hepatocellular carcinoma), human carcinogens (aflatoxin associated with liver cancer, asbestos linked to mesothelioma and vinyl chloride associated with liver angiocarcinoma) and inherited germline cancer susceptibility mutations (BRCA 1 and 2 associated with breast cancer, APC linked to colon cancer). In certain geographical regions, these causative agents account for a significant burden of cancer and may be preventable and/or treatable.

In some embodiments, a set of loci are identified spanning a segment of the genome that does not contain any known tumor suppression genes. In some embodiments a tumor suppressor gene is a gene that encodes a tumor suppressor. In some embodiments, a tumor suppressor is a protein that controls the cell cycle. In some embodiments, a tumor suppressor promotes apoptosis. In some embodiments, a tumor suppressor gene encodes a protein with a regulatory function. In some embodiments, a mutation in a tumor suppressor gene results in loss of a regulatory function and progression toward cancer. In some embodiments, regions containing genes such as but not limited to p53, HNPCC, MEN1, BRCA, Rb, PTEN, VHL, APC, CD95, ST5, YPEL3, ST7 and ST14 which are known tumor suppressor genes would be specifically avoided.

In some embodiments, a set of loci are identified spanning a segment of the genome that does not contain any genes that are causally implicated in carcinogenesis. A list of genes that are causally implicated in carcinogenesis when they become mutated have been described by Futreal et al. in Nature Reviews Cancer (March 2004, Volume 4, 177-183) which is hereby incorporated by reference in its entirety.

In some embodiments, it may be that genotoxin exposed cancers show a higher level of loss of heterozygosity mutational damage across a larger region of the genome, in this case choosing an entire chromosome. Given that driver mutations affecting critical oncogene/tumor suppressor genes are distributed widely across the entire human genome, no single chromosomal region may be said to be entirely exclusive of tumor suppressor genes or oncogenes.

In some embodiments, it is expected that a panel of genotyping markers evenly spanning a chromosome without respect to the location of relevant genes is more likely to be informative of cumulative passenger mutational load.

In some embodiments a set of loci are selected from within chromosome 1, 2, 3, 4, 5, 6, 7, 8, 9, 10, 11, 12, 13, 14, 15, 16, 17, 18, 19, 20, 21 and a combination thereof. In some embodiments a set of loci are selected from within chromosome 2, 3, 4, 5, 6, 7, 8, 10, 11, 12, 13, 14, 15, 16, 18, 19, 20, 21 and a combination thereof. In Some embodiments a set of loci are selected from within chromosome 3, 4, 12, 16 and a combination thereof. In some embodiments, a set of loci are selected from within chromosome 3.

Interest in the role of environmental and occupational chemical exposure as a cause for cancer has recently received increased attention reflected in statements from national oversight bodies and international agencies. The International Association for Research on Cancer (IARC) and other bodies list a wide range of chemical agents as having been proven to be carcinogenic in humans (category 1). Many more compounds are suspected to be capable of cancer formation and await further evidence to establish causality. In attempting to directly link a specific carcinogen or genotoxin with human cancer formation, a common approach is to search for unique mutated gene or locus (or a set of mutations) particular to the agent which caused the cancer. However, such a discriminating signature exists in only a small number of cases; more frequently, the specific mutations are not significantly different between the toxin associated and sporadic cancer subjects. The microscopic cellular appearance of sporadic and toxin associated cancer generally does not show differentiating characteristics. The current lack of tissue based testing to differentiate toxin-associated from sporadic cancer formation can be an important limitation for research regarding cancer causation.

There is now increasing evidence and awareness that environmental and/or occupational exposure to genotoxins such as toxic compounds is causally responsible for a significant proportion of human cancer. The current estimate ranges from 4-7% of the approximately 1.5 million new cancer cases diagnosed each year in the United States being directly attributable to genotoxin exposure. This estimate is generally accepted as being low given that many more cases of cancer are suspected to be of toxin exposure but lack sufficient supportive evidence to affirm causation. This fact has recently been emphasized in the United States by the President's Cancer Panel report as well as by statements from oversight bodies and international agencies. The fundamental challenge facing those working in this area has been a lack of analytic techniques that can discriminate between sporadic versus toxin-associated cancer.

Cancer causation includes well recognized factors such as familial inheritance or germline mutational change involving cancer susceptibility genes and infection with oncogenic viruses and other pathogens. These etiologies can be tested for in cancer tissue to affirm causation and such testing is now accepted as part of routine clinical practice. For most cancers, however, causation remains unclear and its relationship to etiologic factors, such as genotoxins, cannot be easily established except in those instances when toxicologic, epidemiologic and other data is available. Unfortunately, such information is often lacking or not sufficient to affirm causation. Moreover, even when toxicologic and other data is supportive of cancer causation, definitive proof can be lacking to affirm toxin-associated cancer formation. It is towards this large subset of potential genotoxin related or genotoxin-associated cancer subjects that the present embodiments are directed to in an effort to generate molecular data from the cancerous tissues themselves that can assist in the discrimination between sporadic and toxin-associated cancer formation so that a link is more clearly established. Specifically, mutational changes in the DNA of the individual cancer specimen's enables discrimination between sporadic and genotoxin-associated cancer using well characterized subject cohort of genotoxin-exposed and non-exposed individuals.

Chemical agents are capable of directly interacting with and damaging DNA leading to well characterized forms of cancer associated mutational change. In both animal and human studies, the consequences of genotoxic DNA damage have been directly linked to oncogene and tumor suppressor gene mutational change and cancer formation. For specific human cancers, selected chemical toxins produce unique forms of discrete mutational change that is separate and distinct from mutational change occurring in sporadic human cancer of the same type. A DNA signature of mutational change can link chemical exposure with the subsequent development of human cancer. Examples include aflatoxin-induced liver cancer producing point mutation at codon 249 of the TP53 gene and trichloroethylene-induced kidney cancer with point mutation in exon 1 of the von Hippel-Lindau gene, particularly at codon 81. However only a small minority of genotoxin induced human cancer demonstrate such unique signature patterns of DNA damage. In most part, the microscopic changes in the cancer and the pattern of organ/tissue associated oncogenes and tumor suppressor genes are shared irrespective of specific causation. This applies not just to the sporadic versus genotoxin-associated cancer, but for cancers related to inherited gene mutation, oncogenic viruses and other known etiologies. This is not unreasonable since cellular response to carcinogenic stimuli is limited and involvement of cell specific growth regulatory genes is similar irrespective of the factors that initiate cancer development.

Cancer is a multistep process of clonal expansion events that confer progressively greater growth advantage to an affected cell compared to that of unaltered cells. The molecular basis for clonal expansion is DNA damage targeting tissue/organ specific oncogenes and tumor suppressor genes normally responsible for growth regulation. Mutations which result in clonal expansion are termed “driver” mutations in that they are directly responsible for cancer development and progression. The concept of driver mutational change is well accepted and it forms the basis of molecularly targeted chemotherapy to more effectively antagonize those genes directly responsible for specific forms of cancer. However, regions of the genome which do not harbor oncogenes or tumor suppressor genes are also found to be frequently mutated in cancer. These mutations are termed “passenger” mutations, since they do not necessarily drive further neoplastic progression. As a tumor cell population clonally expands, the tumor cells will carry not only driver mutations affecting critical growth regulatory genes but also co-existing passenger mutations that are present in the genome of the affected cell at times of clonal expansion. Chemical agents causing damage to the genome are not known to have specificity for tumor suppressor genes and may be expected to cause widespread genomic damage in an indiscriminate manner, simultaneously producing both driver and passenger mutations.

In some embodiments, passenger mutations present in the genome and targeting noncritical DNA regions will be captured during the clonal expansion process when critical growth regulatory gene mutation occurs and the affected cell undergo a step of neoplastic progression. Thus for each step of clonal expansion, one may expect critical as well as noncritical (passenger) genomic damage. Given that genotoxic DNA damage creates an intense stress on the cell which takes place over a relatively shorter time interval, it is reasonable to hypothesize that a greater degree of passenger mutational change will be present in the genome of toxin induced cancer patients compared to that seen in sporadic cancer patients. In some embodiments, one means to search for such damage is to evaluate a defined genomic region (i.e. a chromosome or chromosomal segment) with particular attention to the extent of accumulated mutational change outside of oncogene/tumor suppressor genes where mutational change would be expected in both sporadic and toxin associated cancer. In yet another embodiment, the greater the extent of passenger mutational change, the more intensely damaged the DNA may be expected to have been at the time when critical cancer gene mutation occurred. In some embodiments, high levels of passenger mutations could then support a mechanism of DNA damage such as occurs in toxin associated cancer formation while a low level of passenger mutations would be more in keeping with sporadic cancer formation.

Some embodiments focus on passenger mutational changes since unlike driver mutations they are not intrinsic to the tumor's development and could therefore serve as a proxy for the intensity and duration of carcinogen exposure, with a higher level of passenger mutations expected for cancers arising under intense chemical exposure than for sporadic cancers. In some embodiments, by searching for passenger mutations, it may be possible to avoid specifically targeting regions with driver genes whose disruption is necessary for cancer progression, since driver mutations would be expected in both sporadic and chlorinated solvent-associated cancer. For example, in sporadic renal cell carcinoma, point mutations in the VHL gene, located on chromosome 3, mutations tend to be distributed widely over the gene's three coding exons, favoring involvement of the distal portion of the coding region (exon 3). By contrast, in trichloroethylene-induced renal cell carcinoma the majority of point mutations in VHL are localized to the distal portion of the gene in exon 1, and in particular a “hot spot” at codon 81 was noted in a sizable minority of patients. Of importance, the trichloroethylene exposed subjects also manifested a higher incidence of tumors with multiple VHL point mutations, a distinctly uncommon occurrence in sporadic RCC. Like other unique toxin-associated mutations, this mutation was found to only be present in toxin-exposed cancers (i.e. specificity of 100%), but was only present in a minority of such cancers (i.e. sensitivity of˜30%). In some embodiments, these additional VHL point mutations can be viewed as increased passenger mutational damage to the gene. A study of the genotoxicity of trichloroethylene and other solvents has found increased DNA fragmentation and formation of micronuclei, which indicate chromosomal instability, a finding consistent with widespread genomic damage.

Based on animal studies and human research, broad classes of chemical agents have been shown to directly damage DNA leading to mutations and cancer. Chlorinated solvents represent one such category of chemical agents capable of DNA adduct formation leading to DNA mismatch pairings and sequence changes of oncogenes and tumor suppressor genes. Renal cell carcinomas (RCC) in particular are known to develop following exposure to high levels of chlorinated solvents and such changes have been described in many forms of toxin associated human cancer.

In a pair of studies of industrial workers exposed to high ambient air levels of trichloroethylene and related chlorinated solvents and sporadic renal cell carcinomas, the investigators found that the microscopic appearance of the kidney cancers in the two cohorts were similar and could not be distinguished from each other. At the DNA level, however, distinct differences were found. Focusing on the von Hippel-Lindau (VHL) gene, known to be closely associated with renal cell carcinoma formation, three patterns in VHL mutations that are different between TCE-exposed and unexposed tumors were identified. First, there was a higher overall incidence of VHL point mutational change in the exposed cohort, in sporadic RCC, VHL point mutations are present in only 30-70% of tumors and the finding of more than a single point mutation is distinctly uncommon. Second, point mutations in the distal portion of exon 1 of the VHL gene were more common in exposed tumors than in sporadic tumors and in particular a ‘hot spot’ site of damage at codon 81 occurred only in exposed tumors. Third, multiple point mutations in the VHL gene were observed in exposed tumors but not in sporadic RCC. The third observation was particularly noteworthy since multiplicity of DNA damage acquisition is uncommon in sporadic RCC but is consistent with relatively more intense DNA damage over a given time period expected from exposure to high levels of a toxin. A similar pattern of increased DNA deletional damage has been demonstrated in asbestos associated non-small cell lung cancer.

Multiplicity of DNA damage in toxin-induced human tumorigenesis, likely reflecting an increased load of passenger mutational change, provided the scientific basis for the approach developed herein. Multiplicity of DNA mutational change were searched in archival tissue specimens from subjects potentially exposed to toxic levels of chlorinated solvents and the findings were compared to that of a control group of individuals with sporadic cancer of the same histologic type.

In some embodiments tissue samples from subjects suspected of having been exposed to a genotoxin form the toxin associated study group. In some embodiments such individuals reside or resided in an area suspected to have high levels of one or more genotoxins in the environment. In some embodiments, the one or more genotoxin may be in the groundwater. In some embodiments, the genotoxin may be present in the ambient air. In another embodiment the genotoxin may comprise an element of the subjects surrounding environment. In another embodiment, genotoxin exposure may be occupational in nature in some embodiments, representative tissue sections of cancer may be obtained from a subset of individuals all of whom willingly provided consent for mutational analysis. In some embodiments a blinded control tumor such as a high grade glioma may be inserted into the series as well.

In some embodiments a separate control cohort of matching type tumors may be obtained from a commercial tissue bank. In some embodiments, the subjects from whom these gliomas are obtained do not report any noteworthy exposure to a particular genotoxin.

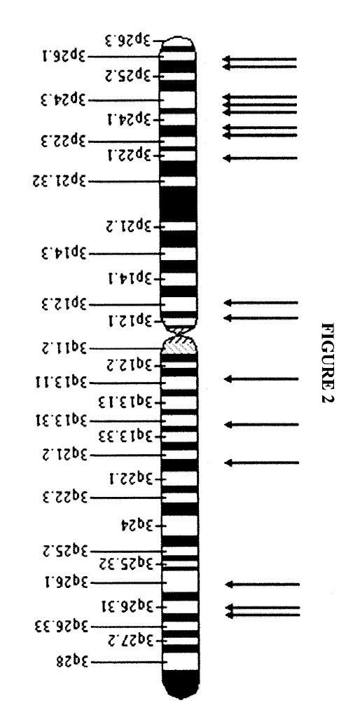

In some embodiments, genomic deletion may be based on the determination of loss of heterozygosity (LOH, allelic imbalance) using polymorphic microsatellite markers randomly distributed across a chromosome. In further embodiments polymorphic microsatellite markers are randomly distributed across chromosome 3. For illustrative purposes, the possible cytogenetic location of a panel of microsatellite markers that may be employed is shown in the chromosome 3 ideogram (FIG. 2). In some embodiments, a panel of microsatellite markers was supplemented with DNA sequencing for TCE-linked point mutations in the distal portion of exon 1 of the VHL gene. For example, where one of the tumors types in the exposure-associated group is a hemangioblastoma and these tumors are associated with von Hippel-Lindau (VHL) syndrome and also have frequent occurrence of VHL mutations in patients without VHL syndrome.

In some embodiments, archival, fixative-treated paraffin-embedded blocks and slides may be reviewed and the diagnosis of cancer confirmed using hematoxylin/eosin staining. In yet other embodiments, representative tissue blocks of each cancer may be re-cut at four micron thickness for microdissection-based mutation analysis. In some embodiments, the histologic growth pattern including level of differentiation may be equivalent across the tumor types and between exposed and control groups.

In further embodiments, after reviewing the histologic slides, specific microscopic cellular targets are selected for tissue microdissection based mutational analysis. In each case, non-neoplastic tissue may be microdissected from non-neoplastic cellular elements to serve as a basis for defining polymorphic site informativeness. In yet other embodiments, the non-neoplastic tissue samples may be taken to be no larger in size than the smallest of the cancer microdissection targets chosen in each individual case and thus contain an equal number or fewer cells than the number of cells in the neoplastic microdissection targets, typically 500 to several thousand. In some embodiments, the non-neoplastic and cancer microdissection tissue targets came from the same slides and may be subject to the same fixation and processing and analyzed in parallel in the laboratory. In some embodiments, the size limitation may ensure that allelic dropout is not mistaken for loss of heterozygosity.

In other embodiments, every non-neoplastic microdissected sample contained an equal number or fewer cells than that present in the neoplastic microdissection targets. This allowed control for allelic dropout given that the non-neoplastic and cancer microdissection tissue targets were subject to the same fixation and processing and analyzed in parallel in the laboratory. The procedures used to perform genotyping have been previously described.

Genomic deletion was based on determination of loss of heterozygosity (LOH, allelic imbalance) using polymorphic microsatellite markers randomly distributed across chromosome 3 (Human Genome Database [www.gdb.org]). The cytogenetic location of the individual microsatellite markers employed in this study is shown in the chromosome ideogram (FIG. 2).

In some embodiments, for loss of heterozygosity (LOH) analysis, microdissected tissue may be PCR amplified with synthetic oligonucleotides bearing fluorescent labels designed for GeneScan fragment analysis (Applied Biosystems). In further embodiments PCR products can be separated by capillary electrophoresis (ABI 3100, Applied Biosystems). In some embodiments, determination of LOH may be based on the peak height ratio of each polymorphic microsatellite performed according to manufacturer's instructions. In some embodiments, post-amplification products may be electrophoresed and relative fluorescence determined for individual allele peak height (GeneScan ABI3100, Applied Biosystems, Foster City, Calif.).

In some embodiments, non-neoplastic microdissected tissue samples may be first evaluated for informative status with respect to individual alleles. In some embodiments, when only a single microsatellite marker peak was found, the patient was designated as non-informative (NI) for that marker.

In some embodiments, for informative markers, the ratio of peaks may be calculated by dividing the value for the shorter sized allele by that of the longer sized allele. In some embodiments, thresholds for significant allelic imbalance may be determined beforehand in extensive studies using normal (non-neoplastic) specimens representing each unique pairing of individual alleles for every marker used in a particular panel. In further embodiments, peak height ratios falling outside of two standard deviations beyond the mean for each polymorphic allele pairing may be assessed as showing significant allelic imbalance. In some embodiments, standardized methods are used to detect and quantify LOH. In further embodiments it is possible to assign a status as being either noninformative, in allelic balance (no LOH) or positive for imbalance (LOH).

In some embodiments, fractional allelic loss (FAL) rate can be calculated as a quantitative measure of acquired LOH mutational change. FAL is defined as the number of markers showing imbalance divided by the total number of informative markers. In some embodiments, FAL enables comparison between different subjects with respect to the degree of cumulative mutational damage even though the loci affected may differ between individuals. In yet other embodiments, the total number of detectable chromosome 3 panel LOH mutations may be evaluated as a measure to differentiate between sporadic versus genotoxin-associated cancer.

In some embodiments, in addition to LOH analysis, the distal half of the first exon of the VHL gene encompassing the trichloroethylene sensitive region and codon 81 hot spot can undergo DNA sequencing in search of point mutational change. In yet other embodiments, the distal exon 1 amplicon can be cycle sequenced by dideoxy chain termination according to the manufacturer's instructions (Applied Biosystems). In yet other embodiments, the analytic sensitivity of the technique is a 10:90 admixture of mutant to wild type alleles.

In some embodiments, data is evaluated for differences in FAL and absolute number of mutations between the two groups via binomial regression. In yet other embodiments, the exposed and non-exposed cohorts were also checked for equivalent numbers of informative markers. In some embodiments, cutoffs in FAL and absolute number of mutations can be estimated to serve as a candidate diagnostic rule for studies of exposed versus unexposed tumors.

In some embodiments, the fractional allelic loss (FAL) rate can be calculated to serve as a quantitative measure of acquired LOH mutational change enabling data from subjects within each cohort to be pooled together for statistical evaluation. In some embodiments, FAL is defined as the number of markers showing imbalance divided by the total number of informative markers. In some embodiments, FAL can be used as a measure to compare different subjects with respect to the degree of cumulative mutational damage. In yet other embodiments, the microdissection target yielding the highest FAL value can represent the subject in the comparative statistical analysis. In yet other embodiments, total number of detectable chromosome 3 LOH mutations, irrespective of informative marker number size, can be evaluated as a measure to differentiate between sporadic versus genotoxin-associated cancer. In yet other embodiments, FAL rate and total passenger mutation number can be used to discriminate thresholds capable of separating two groups or populations.

In some embodiments, potential cutoffs to discriminate toxin-exposed cancers from non-exposed sporadic cancers may be estimated as follows. In some embodiments where there is complete separation between the two groups, the value halfway between the minimum FAL of the exposed group and the maximum FAL of the unexposed group is chosen. In another embodiment, definition of an FAL cutoff is two standard deviations above the mean of the sporadic tumors; this resulted in a cutoff of 0.21. For total number mutations, the smallest number of mutations that is greater than the largest observed number of mutations in the sporadic glioma (control) group; 3 or more mutations (20% of the markers tested, regardless of informative status) defines all tumors in the exposed group is selected.

In some embodiments, multiple distinct LOH events characterize the potential toxin exposed group and such damage could not be accounted for by a single large deletional alteration.

In some embodiments, point mutational change in the distal portion of the first exon of the VHL gene can be searched for. A point mutation at codon 89, in exon 1 of the VHL gene and in proximity to the hot spot site (codon 81) is indicative of a genotoxin-associated cancer. In some embodiments, a sporadic cancer control, inserted as a blinded control will not manifest mutational change in the region of the VHL genome (distal portion of exon 1) associated with chlorinated solvent and genotoxicity-induced carcinogenicity. In some embodiments, the extent of passenger mutational change as a surrogate biomarker for the intensity of widespread genomic damage can be evaluated to detect whether a cancer associated with genotoxin exposure would manifest a significantly higher level of passenger mutations compared to a sporadic cancer. In some embodiments, a panel of polymorphic microsatellite markers is assembled to detect LOH mutational change, the most common cancer-associated DNA structural damage, in a configuration more likely to detect passenger mutations than driver mutations. In some embodiments, the panel of microsatellites is supplemented with DNA sequencing for unique patterns of focused point mutational change (for example, TCE-linked point mutations in the distal portion of exon 1 of the VHL gene). In yet other embodiments, the panel targeted a defined segment of the human genome (for example chromosome 3), but it could equally be applied to other regions of the genome.

In some embodiments, sporadic and toxin-associated cancers, equivalent in histologic appearance, are evaluated using a test population of subjects potentially exposed to significant levels of a genotoxin such as chlorinated solvents and a control group of sporadic tumors of similar histopathologic type. In some embodiments, the analysis also includes blinded sporadic cancer controls revealed after the molecular analysis and mutational profiling interpretation. In some embodiments, Both FAL and total mutation levels will be significantly higher in the toxin-associated cancer cohort compared to a sporadic tumor group. In some embodiments, the common pattern of increased passenger FAL among exposed subjects is consistent with a more widespread damage due to intense genotoxic insult.

In some embodiments, the widespread, non-specific assault by a genotoxin on cellular genomes, cancers in patients who had been exposed to genotoxic agents may demonstrate a higher degree of clonally expanded passenger mutations across a whole chromosome than sporadic tumors.

In some embodiments, increased genomic damage and passenger mutational change is indicative of genotoxin-associated cancer and other toxic related human cancers. Some embodiments are an extension of the approach to a search for widespread genomic passenger mutational damage resulting from exposure to potential chemical carcinogens where traditionally point mutations in a single oncogene or tumor suppressor are analyzed. For example, the limitation of other studies that are focused on a single tumor suppressor such as VHL is that mutational change of the chosen gene is causally responsible for only a defined number of human cancers, in the case of VHL, renal cell carcinoma (RCC) hemangioblastoma, pheochromocytoma and less common human malignancies. While RCC is particularly noteworthy with respect to chlorinated solvent exposure, exposure to toxic levels of such agents has been noted to result in other forms of non-VHL associated human cancer including liver cancer and lymphoma. Given that genomic deletion is the most common type of DNA damage seen in cancer, this DNA alteration was selected for panel analysis rather than single oncogene point mutations because cumulative LOH will serve as a marker for toxin associated cancer formation suitable for application to different forms of human cancer. In another embodiment non-small cell lung cancer arising in subjects with significant levels of asbestos exposure can be evaluated. Such cancers arising in asbestos exposed individuals display a significantly higher rate deletion change to the p16 (CDKN2A) tumor suppressor gene known to act as a driver mutation in lung cancer as well as in many other common and uncommon forms of human malignancy. In some embodiments, toxin associated cancer manifests a significantly higher rate of accumulated deletion damage compared to sporadic cancers of the same type.

In some embodiments, a panel of LOH markers distributed across chromosome 3 is selected as means to detect passenger mutations. However, in some embodiments any genomic DNA segment or combination of segments would have been appropriate for determination of the level of passenger LOH mutations. In further embodiments the LOH search is not restricted only to sites of well characterized loci of oncogene amplification and/or tumor suppressor gene loss since such driver mutations would be present irrespective of causation: sporadic, genotoxin-associated or otherwise.

In some embodiments, the cumulative LOH load differences between toxin exposed and sporadic groups can be accounted for by single large deletional events. In yet other embodiments, The LOH change in tumors from toxin exposed groups are predominantly multiple and discontinuous across the two chromosomal arms of chromosome 3.

Some embodiments provide a means to interrogate cancer specimens for potential causation. In some embodiments, data from larger cohorts evaluating different genomic segments are supported by the results of this study. In some embodiments, it is important to differentiate between cancer causation, from clinical aggressiveness of cancer. Both could be viewed as correlated with high versus low detectable mutation rates. The difference could relate to critical driver mutation rates versus passenger mutation rates. Cancer aggressiveness would correlate with driver mutations directly responsible for clonal expansion whereas widespread genomic damage could be better represented by passenger mutations. In some embodiments, the latter is used to infer the intensity of DNA damage which in turn provides the discrimination between sporadic versus toxin associated cancer causation.

In some embodiments, tumors that may be subjected to the methods described herein include those linked to familial cancers and cancers of indeterminate origin. Also contemplated herein is the differentiation between toxin-induced and sporadic cancers for purposes of cancer treatment. This list is not intended to be exhaustive and can be expanded to include additional old and newly recognized disease states and speaks to the broad applicability of this application to advance toxin-induced versus sporadic cancers and tumors.

Some embodiments are directed to a method of differentiation of non-genotoxin associated versus genotoxin-associated tumor comprising: selecting a set of loci spanning a segment of a genome recognized to harbor no tumor suppressor genes; measuring copy number imbalance at said loci; and counting the number of loci with copy number imbalance, wherein a tumor is determined to be genotoxin-associated when 20% or more of the measured loci show copy number imbalance.

Some embodiments comprise determining the presence of a point mutation in the VHL gene, wherein a point mutation in distal portion of exon 1 is indicative of a genotoxin-associated tumor.

In some embodiments, the distal portion of exon 1 comprises codon 81. Some embodiments comprise determining the presence of point mutation in exons 1, 2 and 3 of VHL gene, wherein the presence of more than one point mutation in said exons is indicative of a genotoxin-associated tumor. In some embodiments, said genotoxin comprises chlorinated solvents, aflatoxin, trichloroethylene, and combinations thereof.

In yet other embodiments, a non-genotoxin associated tumor comprises a sporadic tumor. In yet other embodiments, the genotoxin-associated tumor is from a subject who has a cancer selected from colon, brain, breast, kidney, leukemia, prostate, uterus, stomach, lymphoma, esophagus, sarcoma, thyroid, hemangioblastoma and combinations thereof.

Some embodiments comprise determining passenger fractional allelic loss mutation rate in a segment of a genome. In yet other embodiments, a fractional allelic loss mutation rate of 20% or more is indicative of a toxin associated tumor.

Some embodiments are a method of determining the influence of genotoxin exposure on a tumor and if a tumor's malignant progression has been advanced by genotoxin exposure comprising: counting the number of loci spanning a segment of a genome not containing a tumor suppressor gene with copy number imbalance, wherein a tumor is a genotoxin-associated tumor when about 20% or more of the loci show copy number imbalance.

In yet other embodiments, the said genotoxic compound comprises chlorinated solvents, aflatoxin, trichloroethylene, and combinations thereof. Trichloroethylene (TCE) is a halogenated hydrocarbon widely used for degreasing and other industrial applications, with widespread use beginning in the 1930s and 1940s. It is one of the most common contaminants of groundwater worldwide, and has been found in numerous industrial and military sites across the US and Canada, including at least 852 of the 1430 National Priorities List Superfund sites. Like many other industrial solvents, TCE is known to be genotoxic. Its genotoxicity occurs both through its metabolites, which cause DNA adducts that result in mutations, and via direct exposure to DNA causing frequent strand breaks and other forms of chromosomal instability. TCE has been designated as “carcinogenic to humans by all routes of exposure” by the EPA.

Some embodiments comprise determining the presence of point mutations in the VHL gene. In some embodiments, a point mutation at nucleotide 484 is indicative of a tumor whose malignant progression has been advanced by genotoxic exposure. Some embodiments comprise determining the presence of point mutation in exons 1, 2 and 3 of the VHL gene. In some embodiments, the presence of more than one point mutation in said exons is indicative of a tumor whose malignant progression has been advanced by genotoxic exposure.

In some embodiments the malignancies subjected to the methods described herein include leukemias and lymphomas, and cancers and precancerous conditions that can be readily screened by analysis of a biological specimen from a patient.

In some embodiments, cancers include but are not limited to cancers of the head and neck (e.g., nasal cavity, paranasal sinuses, nasopharynx, oral cavity, oropharynx, larynx, hypopharynx, salivary glands, and paragangliomas), lung tumors (e.g., non-small cell and small cell lung tumors), neoplasms of the mediastinum, brain cancers, cancers of the gastrointestinal tract (e.g., colon, esophageal carcinoma, pancreatic carcinoma, gastric carcinoma, hepatobiliary cancers, cancers of the small intestine, cancer of the rectum, and cancer of the anal region), genitourinary cancers (e.g., kidney cancer, bladder cancer, prostate cancer, cancers of the urethra and penis, and cancer of the testis), gynecologic cancers (e.g., cancers of the cervix, vagina, vulva, uterine body, ovaries, fallopian tube carcinoma, peritoneal carcinoma, and gestational trophoblastic diseases), breast cancer, cancer of the endocrine system (e.g., thyroid tumors, parathyroid tumors, adrenal tumors, pancreatic endocrine tumors, carcinoid tumors, carcinoid syndrome, and multiple endocrine neoplasias), sarcomas of the soft tissues and bone, benign and malignant mesothelioma, skin cancers, liver cancers, malignant melanoma (e.g., cutaneous melanoma and intraocular melanoma), neoplasms of the central nervous system, pediatric tumors (e.g., neurofibromatoses, neuroblastoma, rhabdomyosarcoma, Ewing's sarcoma and peripheral neuroectodermal tumors, germ cell tumors, primary hepatic tumors, and malignant gonadal and extragonadal germ cell tumors), paraneoplastic syndromes, and solids cancers with unknown primary sites. In some embodiments cancer or neoplasm is also meant to include metastatic disease and reoccurrence or relapse of a cancer(s). Also contemplated are virally induced neoplasms such as adenovirus-, HIV-1-, or human papilloma virus-induced neoplasms (e.g., cervical cancer, Kaposi's sarcoma, and primary CNS lymphoma) and any secondary cancer appearing in lymph.

In some embodiments, lymphomas include, but are not limited, to Hodgkin's lymphoma, non-Hodgkin's lymphoma (e.g., B-cell non-Hodgkin's lymphoma and T-cell non-Hodgkin's lymphoma), cutaneous T-cell lymphomas (CTCL), lymphoplasmacytoid lymphoma (LPL), mantle cell lymphoma (MCL), follicular lymphoma (FL), diffuse large cell lymphoma (DLCL), Burkitt's lymphoma (BL), Lennert's lymphoma (lymphoepithelioid lymphoma), Sezary syndrome, anaplastic large cell lymphoma (ALCL), and primary central nervous system lymphomas.

In some embodiments, leukemias include but are not limited to chronic myelogenous leukemia (CML), acute myelogenous leukemia (AML), acute promyelocytic leukemia, acute lymphoblastic leukemia (ALL), prolymphocytic leukemia, hairy cell leukemia, T-cell chronic lymphocytic leukemia, plasma cell neoplasms, chronic lymphocytic leukemia (CLL), and myelodysplastic syndromes (e.g., chronic myelomonocytic leukemia).

In some embodiments, the term cancer can also include aneuploid and diploid cancers, familial and hereditary cancers, virus-induced cancers, chemotherapeutic/radiation-induced cancers, cancers caused by environmental factors, sporadic cancers, and other types indicated herein. Also contemplated is a metastatic cancer, which includes but is not limited to a cancer in any organ presenting as a metastasis but with no apparent primary tumor.

Benign lesions are generally characterized as proliferative or non-proliferative in nature. Non-proliferative lesions are generally not associated with an increased risk of cancer. Proliferative lesions without atypica generally result in a small increase in risk. Atypical hyperplasia is associated with a greater risk of cancer development (i.e., relative risk of about 4 to about 5). Premalignant conditions include, but are not limited to, premalignant organ cancer.

Also contemplated herein is the analysis of cancer recurrence in subjects that may or may not have been exposed to a potential cancer causing toxin. Cancer recurrence includes, but is not limited to, local recurrence after surgery, recurrence after combined surgery and radiation therapy, recurrence after combination treatment of chemotherapy, radiation, surgery, bone marrow transplant, and/or other treatment modalities and combinations thereof.

In some embodiments, the malignancies and precancerous conditions that can be diagnosed using the materials and methods described herein include solid tumors as well as leukemias and lymphomas.

In yet other embodiments, the cancers include but are not limited to cancers of the head and neck (e.g., nasal cavity, paranasal sinuses, nasopharynx, oral cavity, oropharynx, larynx, hypopharynx, salivary glands, and paragangliomas), lung tumors (e.g., non-small cell and small cell lung tumors), neoplasms of the mediastinum, cancers of the gastrointestinal tract (e.g., colon, esophageal carinoma, pancreatic carcinoma, gastric carcinoma hepatobiliary cancers, cancers of the small intestine, cancer of the rectum, and cancer of the anal region), genitourinary cancers (e.g., kidney cancer, bladder cancer, prostate cancer, cancers of the urethra and penis, and cancer of the testis), gynecologic cancers (e.g., cancers of the cervix, vagina, vulva, uterine body, ovaries, fallopian tube carcinoma, peritoneal carcinoma, and gestational trophoblastic diseases), breast cancer, cancer of the endocrine system (e.g., thyroid tumors, parathyroid tumors, adrenal tumors, pancreatic endocrine tumors, carcinoid tumors, carcinoid syndrome, and multiple endocrine neoplasias), sarcomas of the soft tissues and bone, benign and malignant mesothelioma, skin cancers, malignant melanoma (e.g., cutaneous melanoma and intraocular melanoma), neoplasms of the central nervous system, pediatric tumors (e.g., neurofibromatoses, neuroblastoma, rhabdomyosarcoma, Ewing's sarcoma and peripheral neuroectodermal tumors, germ cell tumors, primary hepatic tumors, and malignant gonadal and extragonadal germ cell tumors), paraneoplastic syndromes, and solids cancers with unknown primary sites.

By cancer or neoplasm is also meant to include metastatic disease and reoccurrence or relapse of a cancer(s). Also contemplated are virally induced neoplasms such as adenovirus, HIV-1, or human papilloma virus induced neoplasms such as cervical cancer and Kaposi's sarcoma, and primary CNS lymphoma.

In some embodiments, conditions that create a germ line deletion alteration can be included as having the capacity for dynamic genomic deletion expansion. This would encompass inherited genetic alterations, translocations and inherited or somatically acquired DNA damage.

In some embodiments, lymphomas include but are not limited to Hodgkin's lymphoma, non-Hodgkin's lymphoma (B-cell non-Hodgkin's lymphoma and T-cell non-Hodgkin's lymphoma), cutaneous T-cell lymphomas (CTCL), lymphoplasmacytoid lymphoma (LPL), mantle cell lymphoma (MCL), follicular lymphoma (FL), diffuse large cell lymphoma (DLCL), Burkitt's lymphoma (BL), Lennert's lymphoma (lymphoepithelioid lymphoma), Sezary syndrome, anaplastic large cell lymphoma (ALCL), and primary central nervous system lymphomas.

In some embodiments, leukemias include chronic myelogenous leukemia (CML), acute myelogenous leukemia (AML), acute promyelocytic leukemia, acute lymphoblastic leukemia (ALL), prolymphocytic leukemia, hairy cell leukemia, T-cell chronic lymphocytic leukemia, plasma cell neoplasms, chronic lymphocytic leukemia (CLL), and myelodysplastic syndromes (e.g., chronic myelomonocytic leukemia).

In some embodiments, cancers are those that can be readily screened or found either by visual inspection of the patients skin (skin cancer), mammography (biopsy of lumps), gynecologic cancers (PAP smears and gynecologic examination), or colorectal examination (identification and removal of polyps). The methods and materials provided herein can also be used on tissue samples that have been surgically resected from the patient (resected bowel, removed lung and liver, or other organs).

Various aspects of the present invention will be illustrated with reference to the following non-limiting examples.

### Example 1

Study Cohorts: Tissue from ten brain tumors (9 gliomas comprising both low and high grade disease, and 1 hemangioblastoma) formed the toxin associated study group. These individuals resided in an area suspected to have high levels of chlorinated solvents in the groundwater. Information regarding these subjects and the environmental toxin exposure is available in the public domain. Representative tissue sections of brain cancer were obtained from a subset of these individuals all of whom willingly provided consent for mutational analysis. A blinded control tumor (high grade glioma) was inserted into the series as well. A separate control cohort of 8 brain tumors (7 gliomas and 1 hemangioblastoma) was obtained from a commercial tissue bank. The subjects from whom these gliomas were obtained did not report any noteworthy exposure to chlorinated solvents or other toxic chemicals.

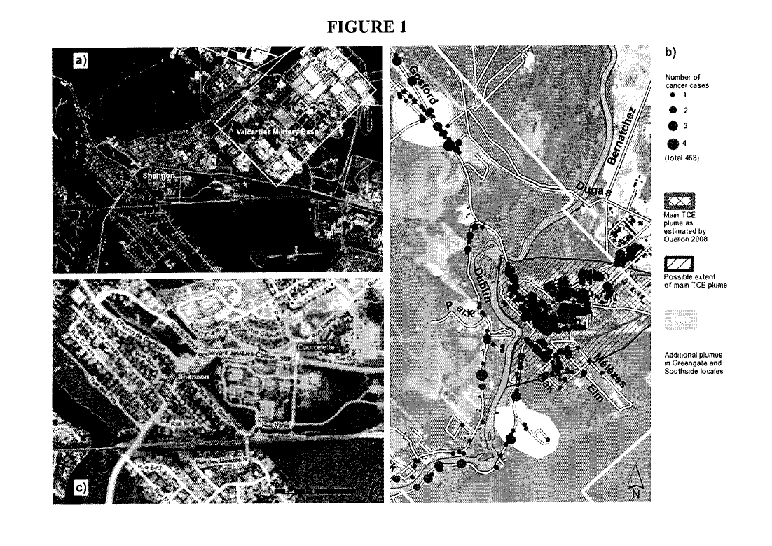

Specimen Analysis: Genomic deletion was based on the determination of loss of heterozygosity (LOH, allelic imbalance) using polymorphic microsatellite markers randomly distributed across chromosome 3 (Human Genome Database). The cytogenetic location of the microsatellite markers employed in this study are shown in the chromosome 3 ideogram (FIG. 1). One of the tumors typoes in the exposure-associated group is a hemangioblastoma, and these tumors are associated with von Hippel-Lindau (VHL) syndrome and also have frequent occurrence of VHL mutations in patients without VHL syndrome. The panel of microsatellite markers was supplemented with DNA sequencing for TCE-linked point mutations in the distal portion of exon 1 of the VHL gene.

Archival, fixative-treated paraffin-embedded blocks and slides were reviewed and the diagnosis of cancer confirmed using hematoxylin/eosin staining. Representative tissue blocks of each cancer were re-cut at four micron thickness for microdissection-based mutation analysis. The histologic growth pattern including level of differentiation was equivalent across the tumor types and between exposed and control groups.

After reviewing the histologic slides, specific microscopic cellular targets were selected for tissue microdissection based mutational analysis. In each case, non-neoplastic tissue was microdissected from non-neoplastic cellular elements to serve as a basis for defining polymorphic site informativeness. The non-neoplastic tissue samples were taken to be no larger in size than the smallest of the cancer microdissection targets chosen in each individual case and thus contained an equal number or fewer cells than the number of cells in the neoplastic microdissection targets, typically 500 to several thousand. Since the non-neoplastic and cancer microdissection tissue targets came from the same slides, they were subject to the same fixation and processing and analyzed in parallel in the laboratory, so the size limitation ensured that allelic dropout was not mistaken for loss of heterozygosity.

For loss of heterozygosity (LOH) analysis, microdissected tissue was PCR amplified with synthetic oligonucleotides bearing fluorescent labels designed for GeneScan fragment analysis (Applied Biosystems). PCR products were separated by capillary electrophoresis (ABI 3100, Applied Biosystems). Determination of LOH was based on the peak height ratio of each polymorphic microsatellite performed according to manufacturer's instructions. Post-amplification products were electrophoresed and relative fluorescence determined for individual allele peak height (GeneScan ABI3100, Applied Biosystems, Foster City, Calif.). Non-neoplastic microdissected tissue samples were first evaluated for informative status with respect to individual alleles. When only a single microsatellite marker peak was found, the patient was designated as non-informative (NI) for that marker.

For informative markers the ratio of peaks was calculated by dividing the value for the shorter sized allele by that of the longer sized allele. Thresholds for significant allelic imbalance were determined beforehand in extensive studies using normal (non-neoplastic) specimens representing each unique pairing of individual alleles for every marker used in the panel. Peak height ratios falling outside of two standard deviations beyond the mean for each polymorphic allele pairing were assessed as showing significant allelic imbalance. The standardized methods used to detect and quantify LOH have been previously reported. Thus, it was possible to assign a status as being either noninformative, in allelic balance (no LOH) or positive for imbalance (LOH).

The fractional allelic loss (FAL) rate was then calculated as a quantitative measure of acquired LOH mutational change. FAL was defined as the number of markers showing imbalance divided by the total number of informative markers. FAL enables comparison between different subjects with respect to the degree of cumulative mutational damage even though the loci affected may differ between individuals. Likewise, the total number of detectable chromosome 3 panel LOH mutations was evaluated as a measure to differentiate between sporadic versus genotoxin-associated cancer.

In addition to LOH analysis, the distal half of the first exon of the VHL gene encompassing the trichloroethylene sensitive region and codon 81 hot spot underwent DNA sequencing in search of point mutational change. The distal exon 1 amplicon was cycle sequenced by dideoxy chain termination according to manufacturer's instructions (Applied Biosystems). The analytic sensitivity of the technique is a 10:90 admixture of mutant to wild type alleles.

Statistical Analysis: The data was evaluated for difference in FAL and absolute number of mutations between the two groups via binomial regression. The exposed and non-exposed cohorts were also checked for equivalent numbers of informative markers. Cutoffs in FAL and absolute number of mutations were estimated to serve as a candidate diagnostic rule for further studies of exposed versus unexposed tumors.

Results: The fractional allelic loss (FAL) rate and level of total detectable mutations for the chlorinated solvent exposed and control cohorts are shown in Tables 1 and 2.

TABLE 1

| CYT DIST | | | | | | | | |
|---|---|---|---|---|---|---|---|---|
| P26.1 | P25.3 | P24.3 | P24.2 | P24.2 | P23 | P22.1 | P12.3 | P12.1 |
| STS ID | | | | | | | | |
| 40803 | 147895 | 149055 | 505233 | 149354 | 51439 | 49304 | 55484 | 149264 |
| DIST TO P-TERM | | | | | | | | |
| 1.04 | 10.34 | 17.93 | 25.3 | 25.55 | 33.22 | 42.79 | 78.58 | 87.92 |

CHLORINATED SOLVENT EXPOSED GLIOMAS

| | | | | | | | | | | |
|---|---|---|---|---|---|---|---|---|---|---|
| 1 | GLIOMA, LG | NO | NI | NO | NO | NI | NO | LOH | NO | NI |
| 2 | GLIOMA, HG | NI | LOH | NO | LOH | NI | NI | NO | NI | NO |
| 3 | GLIOMA, HG | NO | LOH | LOH | NO | NO | NI | NI | NI | LOH |
| 4 | GLIOMA, LG | LOH | NO | NI | NI | NI | NI | LOH | NI | NO |
| 5 | GLIOMA, HG | NI | NO | NI | LOH | NI | NI | NI | LOH | NI |
| 6 | GLIOMA, LG | NO | NI | NI | NI | LOH | NO | NI | LOH | NI |
| 7 | GLIOMA, HG | NI | NO | NI | LOH | NO | NI | NI | NI | NI |
| 8 | GLIOMA, LG | NO | NO | LOH | NO | LOH | NI | NO | LOH | NO |
| 9 | GLIOMA, HG | NI | NI | NO | LOH | NO | NI | NI | NI | NI |

BLINDED SPORADIC CONTROLS

| | | | | | | | | | | |
|---|---|---|---|---|---|---|---|---|---|---|
| 10 | GLIOMA | LOH | NO | NO | NI | NO | NO | NO | NI | NI |

SPORADIC GLIOMA

| | | | | | | | | | | |
|---|---|---|---|---|---|---|---|---|---|---|
| 11 | GLIOMA, LG | NO | NI | NO | NO | NO | NO | NO | NO | NO |
| 12 | GLIOMA, HG | NO | NO | NI | NI | NO | NO | NO | NO | NI |
| 13 | GLIOMA, HG | NO | NI | NO | NI | NI | NO | NO | NO | NI |
| 14 | GLIOMA, LG | LOH | NO | NO | NI | NO | NI | NI | NO | NI |
| 15 | GLIOMA, GBM | NI | NO | NI | NI | NO | NO | NO | NO | NO |
| 16 | GLIOMA, GBM | NO | NO | NO | NO | NO | NO | NI | NO | NI |

| | | CYT DIST | | | | | | | |
|---|---|---|---|---|---|---|---|---|---|
| | | Q13.11 | Q13.31 | Q21.3 | Q22.1 | Q26.1 | Q26.31 | | |
| | | STS ID | | | | | | | |
| | | 78477 | 29910 | 149337 | 42191 | 32667 | 149366 | | |
| | | DIST TO P-TERM | | | | | | | |
| | | 105.24 | 116.31 | 128.38 | 134.17 | 163.16 | 173.18 | FAL | |
| CHLORINATED SOLVENT EXPOSED GLIOMAS | | | | | | | | | |
| 1 | GLIOMA, LG | NI | NO | NI | NI | LOH | LOH | 3/9 | 0.33 |
| 2 | GLIOMA, HG | LOH | LOH | LOH | LOH | NI | NO | 6/10 | 0.6 |
| 3 | GLIOMA, HG | NO | NI | NO | LOH | NI | NI | 4/9 | 0.44 |
| 4 | GLIOMA, LG | NI | LOH | NI | NI | NI | NI | 3/5 | 0.6 |
| 5 | GLIOMA, HG | NI | LOH | LOH | NI | NO | NI | 4/6 | 0.67 |
| 6 | GLIOMA, LG | LOH | NI | LOH | NO | NI | NI | 4/7 | 0.57 |
| 7 | GLIOMA, HG | LOH | NI | NI | LOH | NI | NI | 3/5 | 0.6 |
| 8 | GLIOMA, LG | NI | NO | NO | NI | LOH | LOH | 5/12 | 0.42 |
| 9 | GLIOMA, HG | LOH | LOH | LOH | NO | NI | LOH | 5/8 | 0.63 |
| BLINDED SPORADIC CONTROLS | | | | | | | | | |
| 10 | GLIOMA | NO | NO | LOH | NI | NO | NO | 2/11 | 0.18 |
| SPORADIC GLIOMA | | | | | | | | | |
| 11 | GLIOMA, LG | NI | NI | NI | LOH | NI | NO | 1/10 | 0.1 |
| 12 | GLIOMA, HG | NO | NI | NI | NI | NI | NO | 0/8 | 0 |
| 13 | GLIOMA, HG | NO | NO | NI | LOH | NI | NI | 1/8 | 0.13 |
| 14 | GLIOMA, LG | NO | NO | NO | NO | NI | NI | 1/9 | 0.11 |
| 15 | GLIOMA, GBM | LOH | NO | NI | NO | NO | NI | 1/10 | 0.1 |

| | | | | | | | | |
|---|---|---|---|---|---|---|---|---|
| 16 | GLIOMA, GBM | NI | NO | NI | NO | LOH | NI | 1/10/0.1 |

Abbreviations.
NI = noninformative status for the particular microsatellite marker,
NO = no LOH detected,
LOH = loss of heterozygosity detected (see methods for details),
MSI = microsatellite instability present precluding LOH analysis.
LG = Low grade,
HG = High grade,
GBM = Glioblastoma multiforme.

TABLE 2

| | | FAL | | Number of Mutations | |
|---|---|---|---|---|---|
| | | | Std | | Std |
| | Subjects | Average | Dev | Average | Dev |
| EXPOSED GLIOMA | 9 | 0.54 | 0.11 | 4.1 | 1.0 |
| SUBJECTS | | | | | |
| SPORADIC | 7 | 0.09 | 0.06 | 0.9 | 0.6 |
| GLIOMAS | | | | | |

FAL represents fractional allelic loss rate (see methods for description). Total mutations represents the detectable mutations for each cancer irrespective of the level of informative markers.

Compared to the 7 subjects with sporadic gliomas (control group), the 9 chlorinated solvent-exposed gliomas (exposed group) manifested a significantly higher FAL: average FAL of 0.54+/−0.11 for the exposed group vs. 0.09+/−0.06 for the control group, p=4.13e-07. Similarly, the absolute number of detectable LOH mutations in the exposed glioma group was significantly higher [compared to the sporadic cancer control gliomas: 4.1+/−1.0 mutations for the exposed group vs. 0.9+/−0.6 mutations for the control group, p=4.13e-07. There was not a statistically significant difference in the number of informative markers between the exposed [7.9+/−2.4 markers] and control cases [9.4+/−1.1 markers] (p=0.14).

Potential cutoffs to discriminate toxin-exposed cancers from non-exposed sporadic cancers were estimated as follows. For an FAL cutoff, since there was complete separation between the two groups, the value halfway between the minimum FAL of the exposed group and the maximum FAL of the unexposed group is chosen. This resulted in a FAL cutoff of 0.26. Another definition of an FAL cutoff is two standard deviations above the mean of the sporadic tumors; this resulted in a cutoff of 0.21. For total number mutations, the smallest number of mutations that is greater than the largest observed number of mutations in the sporadic glioma (control) group; 3 or more mutations (20% of the markers tested, regardless of informative status) defines all tumors in the exposed group is selected (Table 2).

In addition of quantitative differences in the level of detectable LOH changes, all gliomas in the exposed group shows LOH events on both chromosomal arms (Table 1). When multiple LOH events were detected on the same chromosomal arm involving nonadjacent markers, the absence of LOH in intervening informative markers along the chromosome affirmed that a single deletion event could not account for all the LOH changes (Table 1). The 10 gliomas arising in potentially exposed subjects displayed multiple p_arm_LOH change in 5 cases of which only 1 could be attributed to a single large chromosomal deletion. The 10 gliomas subjects showed 2 tumors with multiple detectable LOH mutations in which one could possibly be ascribed as a single large chromosomal deletion. Each case had a minimum of 2 distinct events, and at least 6 cases had multiple discrete LOH events within a chromosome arm. Thus multiple distinct LOH events characterized the potentially toxin exposed group and such damage could not be accounted for a single large deletional alteration.

Point mutational change in the distal portion of the first exon of the VHL gene was searched for in all the cases. A point mutation at codon 89, in exon 1 of the VHL gene and in proximity to the hot spot site (codon 81) was detected in a hemangioblastoma subject from the toxin associated subject. A sporadic control hemangioblastoma, inserted as a blinded control did not manifest mutational change in the region of the VHL genome (distal portion of exon 1) associated with chlorinated solvent carcinogenicity (FIG. 2). None of the gliomas, neither sporadic nor toxin exposed, showed VHL gene point mutation.

The extent of passenger mutational change as a surrogate biomarker for the intensity of widespread genomic damage was evaluated to detect whether a cancer associated with genotoxin exposure would manifest a significantly higher level of passenger mutations compared to a sporadic cancer. A panel of polymorphic microsatellite markers were assembled to detect LOH mutational change, the most common cancer-associated DNA structural damage, in a configuration more likely to detect passenger mutations than driver mutations. The panel of microsatellites was supplemented with DNA sequencing for unique patterns of focused point mutational change (TCE-linked point mutations in the distal portion of exon 1 of the VHL gene). The panel targeted a defined segment of the human genome (chromosome 3), but it could equally have been applied to other regions of the genome. Sporadic and toxin-associated brain cancers, equivalent in histologic appearance, were evaluated using a test population of subjects potentially exposed to significant levels of chlorinated solvents and a control group of sporadic brain tumors of similar histopathologic type. The analysis also included blinded sporadic cancer controls revealed after the molecular analysis and mutational profiling interpretation. Both FAL and total mutation levels were significantly higher in the toxin associated brain cancer cohort compared to the sporadic brain tumor group (Table 2). The common pattern of increased passenger FAL among exposed subjects is consistent with a more widespread damage due to intense genotoxic insult.

The results of this study demonstrate increased genomic damage and passenger mutational change in chlorinated solvent associated cancer and other toxic related human cancers. This study extended the approach to a search for widespread genomic passenger mutational damage resulting from exposure to potential chemical carcinogens. The limitation of other studies that are focused on a VHL based approach is that mutational change of this gene is causally responsible for only a defined number of human cancers, most notably RCC but also including hemangioblastoma, pheochromocytoma and less common human malignancies. While RCC is particularly noteworthy with respect to chlorinated solvent exposure, exposure to toxic levels of such agents has been noted to result in other forms of non-VHL associated human cancer including liver cancer and lymphoma. Given that genomic deletion is the most common type of DNA damage seen in cancer, this DNA alteration was selected for panel analysis rather than VHL point mutation with the expectation that cumulative LOH would serve as a marker for toxin associated cancer formation suitable for application to different forms of human cancer.

In an analogous study of non-small cell lung cancer arising in subjects with significant levels of asbestos exposure, such cancers arising in asbestos exposed individuals displayed a significantly higher rate deletion change to the p16 (CDKN2A) tumor suppressor gene known to act as a driver mutation in lung cancer as well as in many other common and uncommon forms of human malignancy. Accordingly, toxin associated cancer manifests a significantly higher rate of accumulated deletion damage compared to sporadic cancers of the same type.

A panel of LOH markers distributed across chromosome 3 (Table 1) were selected as means to detect passenger mutations. However, any genomic DNA segment or combination of segments would have been appropriate for determination of the level of passenger LOH mutations. It is, however, important not to narrow the LOH search only to sites of well characterized loci of oncogene amplification and/or tumor suppressor gene loss since such driver mutations would be expected to be present irrespective of causation: sporadic, genotoxin-associated or otherwise.

Of note here is the observation that the cumulative LOH load differences between toxin exposed and sporadic groups could not be accounted for by single large deletional events. The LOH change in tumors in the exposed group were predominantly multiple and discontinuous across the two chromosomal arms of chromosome 3.

The results of this study provide support that a molecular approach on potentially exposed tissue is both feasible and likely to be productive at discriminating the role of toxic chemical exposure. The importance of correlating toxicologic data on exposure and corresponding molecular changes cannot be overemphasized. The results described here provide support that such an approach will enable better understanding and linking the two independent sources of cancer related information.

The results of this study provide a means to interrogate cancer specimens for potential causation. Data from larger cohorts evaluating different genomic segments are supported by the results of this study. Finally, it is important to differentiate between cancer causation, from clinical aggressiveness of cancer. Both could be viewed as correlated with high versus low detectable mutation rates. The difference could relate to critical driver mutation rates versus passenger mutation rates. Cancer aggressiveness would correlate with driver mutations directly responsible for clonal expansion whereas widespread genomic damage could be better represented by passenger mutations. The latter is used here to infer the intensity of DNA damage which in turn provides the discrimination between sporadic versus toxin associated cancer causation.

### Example 2

Some embodiments are a method for differentiating genotoxin-associated cancer from sporadic cancer based on the quantitative level of “passenger” DNA mutations. In some embodiments, it is applicable to many forms of cancer and a variety of archived, fixative-treated clinical tissue specimens. The present example is an application of this method to a group of 27 subjects who were exposed to trichloroethylene via groundwater contamination and who have developed a variety of cancers including colon, brain, breast, kidney, and other cancers, compared to 10 individuals with sporadic (unexposed) cancers without known exposure to chlorinate solvents as controls. The control cases consisted of two types: 1) cancers from subjects residing in the general vicinity of the exposed subjects, but outside the region of high groundwater contaminant levels, 2) a separate group of cancers from a tissue bank. All controls were analyzed in the same way as the exposed subjects. The total number of passenger LOH mutations and the passenger fractional allelic loss (FAL) index were determined for the sporadic and genotoxin-exposed cancer cohorts. The population in Shannon, Quebec has well-characterized environmental exposure; among this group passenger FAL correlated strongly with exposure, with exposed cancers showing distinctly greater passenger FAL than sporadic cancer. Classification thresholds were tested for total number of mutations and FAL and these separate genotoxin from sporadic cancers nearly completely. This molecular approach can be applied to diverse forms of human cancer to distinguish between sporadic and genotoxin-associated cancers, and may provide a new, independent means to assist in establishing cancer causality.

The town of Shannon, Quebec, located approximately 25 km north and east of the city of Quebec in Quebec, Canada, lies adjacent to the Canadian Defense Forces Valcartier military base. As on many military bases, extensive use of was made of TCE from the 1940s until as late as 2000. During this period, much of this TCE was disposed of in unlined pits (i.e., locations accessible to groundwater). The TCE made its way into the groundwater of the region, and formed a plume of contamination which stretched west across the base, arriving at the town of Shannon between 1956 and 1970. Extensive hydrologic and toxicologic research on the region and the plume conducted from 1997 onward made numerous measurements of the extent of the plume and concentrations of TCE in the groundwater: The groundwater plume migrating to the west within the Valcartier base had a maximum concentration of 1200 ug/L in 2001, but most of the high concentrations vary between 560 and 920 ug/L in the core of the dissolved TCE plume within the Valcartier base. At the property boundary between the base and the town of Shannon, maximum concentrations measured in 2001 were from 260 to 340 ug/L. Maximum concentrations observed within the base in 2001 were greater than 4500 ug/L, with a peak concentration of 13500 ug/L.

The migration of the plume to the west caused contamination of potable water supplies of many residents of Shannon. Extremely and persistently high concentrations of TCE were found within a particular area of Shannon; this area has been termed the “Red Zone”, and is shown in FIG. 1. Measurements between 1999 and 2001 showed TCE levels in wells of the red zone area ranging from 800 to 1200 microg/L, i.e. up to 240 times higher than the 5 microg/L Health Canadian guidelines. Levels prior to 2001 are alleged to have been much higher and the size of the plume larger, this was confirmed by a 2008 toxicologic study of cancer in the Red Zone, which showed an average of 214 ug/L in these wells. Other residents of Private Married Quarters on the base were also exposed to extremely high levels of TCE via water from a contaminated well on the base.

In other areas of Shannon, groundwater providing potable water to residents was also contaminated, though not at levels as persistently high as within the Red Zone. Thus the overall exposure to TCE of a Shannon resident was governed in substantial part by the residence address of that person. Of course residents, both inside and outside the Red Zone would have also been exposed to TCE from sources outside their residence, such as potable water at their workplace, the homes of friends and colleagues, etc., so that residential address cannot be considered a perfect proxy for exposure.

A toxicology study in 2008 examined the levels of TCE exposure and incidence of cancer in 55 homes within the Red Zone, and compared these to homes in Shannon with little or no TCE contamination, with the aim of examining the risk of cancer due to TCE in a comparable population (i.e. residents of Shannon). The study found that those residing in the Red Zone were at approximately 5 times the risk of cancer as those with limited TCE in their potable water, and concluded that the increased risk was linked to the TCE exposure.

Shannon provides a unique circumstance to examine cancer causation and environmental exposure to genotoxic agents due to a confluence of factors: a contained geographic region with a well-known contaminant where hydrology and toxicology have been thoroughly characterized and well-defined population of residents manifesting a statistically significant cancer cluster.

Some embodiments focus on the task of discriminating existing cancers according to their association with exposure to TCE and similar chlorinated solvents.

MATERIALS AND METHODS: Tumor tissue was available from a cohort of exposed and unexposed residents of the Shannon Quebec area, as well as cancers from a tissue bank. The exposed cohort consisted of 27 residents of Shannon or the Private Military Quarters of the Valcartier base with demonstrated environmental exposure to TCE. The unexposed cohort consisted of 6 tissue bank cancers plus four controls from the Shannon/Quebec City region. For all the exposed subjects and the controls from Shannon/Quebec city molecular analysis was performed blind to the subjects exposure status.

The subjects from whom tissue bank specimens were obtained did not report an exposure to chlorinated solvents or other toxic chemicals. While this would not exclude possible (though likely limited) toxic exposure, any exposure would form part of a baseline measurement for sporadic glioma patients. Tobacco use was ascertained via self-reporting from subjects or next of kin for all exposed subjects and two unexposed subjects, and was unavailable for eight unexposed subjects. Table 3 shows demographics, smoking status, residence and duration of exposure and exposure grade for all subjects.

To characterize exposure for analysis each subject was assigned a semi-quantitative exposure grade ranging from 0 to +3 reflecting the duration and intensity of exposure to chlorinated solvents. The exposure grade was based primarily on residential location, with the Red Zone (see FIG. 1) and other locations where wells showed persistently high levels of TCE in water being assigned the highest grade and those with no exposure being assigned the lowest. To conservatively represent exposure, we chose the period from 1980 to 2005 as the reference interval for exposure. Tissue bank controls and control subjects from outside of Shannon were assigned a grade of 0. Subjects residing in locations with limited contamination were assigned a grade of 0 to 1, those with greatest exposure were assigned a grade of 3, and the remainder were assigned grades of 1 or 2 to 3 to account for limited knowledge in the absolute degree of exposure. The ranges of exposure data arise from the fact that people may have worked or socialized in different areas of Shannon or elsewhere and thus suffered differing exposures, and as is common in environmental exposure studies, a precise numerical exposure score is neither feasible nor reliable.

SPECIMEN ANALYSIS: Archival, fixative-treated paraffin-embedded blocks and slides were reviewed and the diagnosis of cancer confirmed using hematoxylin/eosin staining. Representative tissue blocks of each cancer were recut at four microns thickness for microdissection based mutation analysis. The histologic growth pattern including level of differentiation was equivalent across the tumor types and between exposed and control groups.

After histologic review of the slides, specific microscopic cellular targets were selected for microdissection-based mutational analysis. In each case, non-neoplastic tissue was microdissected from non-neoplastic cellular elements to serve as a basis for defining polymorphic site informativeness. The non-neoplastic tissue samples were taken to be smaller in size than the smallest of the cancer microdissection targets chosen in each individual case and thus contained an equal number or fewer cells than that present in the neoplastic microdissection targets. Since the non-neoplastic and cancer microdissection tissue targets were subject to the same fixation and processing and analyzed in parallel in the laboratory, having more DNA from tumor regions ensured that allelic dropout would not be mistaken for LOH.

Genomic deletion was based on determination of loss of heterozygosity (LOH, allelic imbalance) using polymorphic microsatellite markers reasonably evenly distributed across chromosome 3 (Human Genome Database [www.gdb.org]). The cytogenetic location of the individual microsatellite markers employed in this study is shown in the chromosome ideogram in FIG. 2.

For loss of heterozygosity (LOH) analysis, microdissected tissue was PCR amplified with synthetic oligonucleotides bearing fluorescent labels designed for GeneScan fragment analysis (Applied Biosystems). PCR products were separated by capillary electrophoresis (ABI 3100, Applied Biosystems). Determination of LOH was based on the peak height ratio of polymorphic microsatellite performed according to manufacturer's instructions. Post-amplification products were electrophoresed and relative fluorescence determined for individual allele peak height (GeneScan ABI3100, Applied Biosystems, Foster City, Calif.). Non-neoplastic microdissected tissue samples were first evaluated for informative status with respect to individual alleles. When only a single microsatellite marker peak was found, the patient was designated as non-informative (NI) for that marker.

For informative markers the ratio of peaks was calculated by dividing the value for the shorter sized allele by that of the longer sized allele. Thresholds for significant allelic imbalance were determined beforehand in extensive studies using normal (non-neoplastic) specimens representing each unique pairing of individual alleles for every marker used in the panel. Peak height ratios falling outside of two standard deviations beyond the mean for each polymorphic allele pairing were assessed as showing significant allelic imbalance. The standardized methods used to detect and quantify LOH have been previously reported. Thus it was possible to assign a status as being either noninformative, in allelic balance (no LOH) or positive for imbalance (LOH).

For each specimen, the fractional allelic loss (FAL) rate was calculated as a quantitative measure of acquired LOH mutational change enabling data from subjects within each cohort to be pooled together for statistical evaluation. FAL was defined as the number of markers showing imbalance divided by the total number of informative markers. The measure has been used in our previous work and is also accepted as a measure to compare different subjects with respect to the degree of cumulative mutational damage. The microdissection target yielding the highest FAL value represented the subject in the comparative statistical analysis. Also total number of detectable chromosome 3 LOH mutations, irrespective of informative marker number size, was also evaluated as a measure to differentiate between sporadic versus genotoxin-associated cancer.

STATISTICAL ANALYSIS: The data was evaluated for difference in FAL and absolute number of mutations via binomial regression, using exposure (binary, exposed vs. unexposed) as the independent variable. Similarly, duration of exposure and exposure grade were tested for difference in FAL. Because age, gender and smoking status were not available for most of the unexposed, these could not be evaluated as covariates in the regression. Nonetheless, correlations between FAL and these variables (age, gender, smoking status) were evaluated for all exposed cases.

The exposed and non-exposed cohorts were also checked for equivalent numbers of informative markers. Cutoffs in FAL and number of mutations established in previous work were evaluated for their ability to classify exposed and unexposed cases.

RESULTS: Genomic location of LOH mutations and the number and FAL rate for each patient are shown in Table 4. The pattern of increased numbers of mutations in the exposed group can readily be distinguished, and reflects the extent of passenger mutational change. While chromosome 3 contains some growth regulatory genes, the random pattern of observed mutations supports passenger genomic damage rather than mutations affecting of a particular cancer-associated gene.

One subject with colon cancer showed extensive instability of microsatellites in keeping with DNA repair gene mutational change (Table 3, case 7). Family history information was available on this subject with no support for familial cancer susceptibility (Lynch syndrome), and thus DNA repair gene mutation was deemed to be a somatic rather than inherited event. The observation of microsatellite instability in 11 of 13 informative markers precluded determination of LOH, so FAL was not calculated for this patient.

The 26 exposed tumors without MSI manifested a significantly higher FAL than the 10 unexposed (Table 5) via binomial regression (p=2.6e⁻⁹). Similarly, correlations between FAL and exposure duration (p=0.01, Pearson correlation coefficient) and overall grade (p=5.5e⁻⁵) were also found. However, the correlations with grade and duration likely reflect exposure vs. non-exposure; among the exposed group only, there was not a significant correlation between either the semi-quantitative grade of exposure or exposure duration (p=0.79). This is not a surprising finding given the number of subjects and the variation inherent in environmental exposure. There was no statistically significant difference in the number of informative markers between the exposed and control cases (p=0.22).

Smoking history was captured for 28 subjects as “whether the subject had ever been a regular smoker”, with 16 ever-smokers and 12 never-smokers. Smoking was not significantly correlated with FAL (p=0.48). Since smoking status missing for many of the unexposed subjects, this lack of correlation primarily applies to the exposed subjects. However, these results do not support smoking as a primary driver of the observed difference in FAL between exposed and unexposed subjects. Age at first cancer diagnosis was available for 28 subjects, with an average of 49.6 It was also not significantly correlated with FAL (p=0.55), with similar limitations of missing values for many of the unexposed.

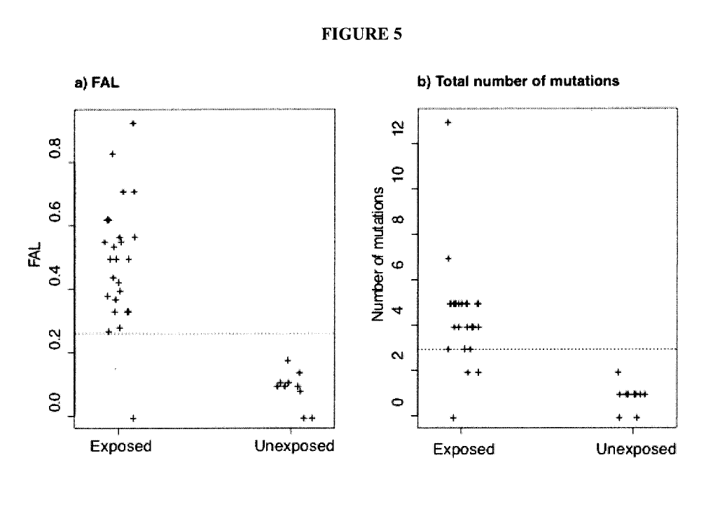

A cutoff of FAL≧0.26 was established to predict toxin exposure versus sporadic cancer formation (6). Applying this cutoff to the present cohort correctly predicts exposure status for all but one false negative exposed case (FIG. 5), yielding a sensitivity of 96% [95% CI: 80-100%], a specificity of 100% [95% CI: 69-100%], and an overall accuracy of 97% [95% CI: 85-100%]. For absolute numbers of mutations, the previous cutoff of >=3 mutations was evaluated, yielding slightly lower accuracy of 92% [95% CI: 78-98%]. The effectiveness of this diagnostic rule adds support to the idea of interrogating passenger mutations on chromosome 3 as a means for distinguishing sporadic from toxic exposure-associated tumors.

Both renal cell carcinoma subjects, one from the exposure zone and one from outside the zone, underwent DNA sequencing of exon 1 of the VHL gene, encompassing the hot spot region previously reported to be closely associated with trichloroethylene associated carcinogenesis. Neither kidney cancer showed any point mutations in VHL exon1. While hot spot region point mutation is closely associated with trichloroethylene exposure, not all exposed subjects show this change.

This study examined whether an elevated level of passenger mutational damage could be used to differentiate between sporadic cancer versus genotoxin associated cancer. While passenger mutational change is not restricted to toxin-associated cancer, a high passenger FAL mutational rate would be consistent with more intense DNA damage associated with intense genotoxic assault. Examining this widespread damage could potentially differentiate between sporadic (less intense accumulation of mutations) versus toxin-associated cancer formation.

Using the passenger mutation FAL methodology, we evaluated a cohort of 27 cancer subjects who lived in the Shannon, Quebec region for a period of no less than 5 years at a time when groundwater and drinking water were contaminated with chlorinated solvents. We compared these 27 individuals to 10 control cancer subjects who did not have this exposure, seeking differences in the number and or distribution of LOH mutations between these two groups. This study primarily evaluated four types of cancer in both groups: 1) colon cancer, in light of its exceptionally high level in the Shannon red zone; 2) brain cancer (glioma), as this uncommon form of human cancer was notably present in the exposed population; 3) breast cancer, a common form of sporadic cancer; and, 4) kidney cancer, as this type of malignancy has been closely associated with exposure to chlorinated solvents. The different forms of cancer served to test the hypothesis that determination of the number of passenger mutations could discriminate between different histologic forms of sporadic and genotoxin-associated cancers across a variety of tumor types.

While this study examined chromosome 3 for passenger FAL and total mutation rate, it need not be the only site for interrogation to determine the rate of such damage. There is evidence that chemical carcinogenesis induces widespread genomic damage and it is reasonable to hold that such damage would be incorporated at the time of clonal expansion when driver growth regulatory gene damage affecting specific growth regulatory genes leads to neoplastic progression. However, for any region examined, it is essential to distinguish between driver mutations involving critical growth regulatory genes from passenger mutational damage that involves genomic regions not likely to result in growth deregulation when subject to mutational change. In some embodiments, the methodology described here may be designed to detect the degree of nonspecific genome wide passenger mutational change independent of specific cell cycle regulatory genes, and therefore should be broadly applicable to a spectrum of cancer types. This is supported by the random distribution of LOH present within and across different forms of cancer in this study cohort (Table 3).

Multistep clonal expansion is critical to this analysis since each clonal expansion step fixes DNA mutational damage in the genome whether such damage affects critical growth regulatory genes or involves non-critical regions of the genome. A high passenger mutational FAL rate supports intense and persistent genomic damage which is consistent with but not necessarily restricted that resulting from genotoxic chemical exposure. A low passenger FAL mutational rate would support a slow rate of DNA damage more consistent with sporadic cancer formation. Integrating the passenger FAL rate analysis with other independent parameters of cancer offers the best potential to discriminate between causations.

Alternative etiologies for cancer associated with more intense damage and more rapid accumulation of mutations could be expected to yield high passenger FAL rates and high total number of marker panel LOH mutations as seen here. For example, patients with inherited DNA repair gene mutations could exhibit and equivalent high passenger FAL rate as that of the exposed subjects in this study. In DNA repair gene deficient patients, the rate of DNA damage may be relatively low as would occur in sporadic cancer patients however the repair deficiency could lead to there being a higher rate of passenger mutational change when a clonal expansion event supervenes. None of the toxin-exposed subjects reported a family history of cancer susceptibility.

Though some toxic-exposed tumors demonstrate mutations of specific genomic loci, this type of mutational change is uncommon and therefore not especially useful when studying the majority of suspected chemically induced human cancer subjects. Aside from VHL mutation linked human cancer (renal cell carcinoma, hemangioblastoma, pheochromocytoma), point mutation of the VHL gene would not be expected in other malignancies because a mutation in this gene would not necessarily confer growth advantage, and would be no different from random damage to be expected in any region of the genome not bearing cell specific growth regulatory genes involved in carcinogenesis.

A cutoff of FAL≧0.26 discriminated nearly all exposed from unexposed tumors, suggesting that this methodology provides a technique to test routine tumor specimens for exposure to chlorinated solvents and other genotoxins causing similar patterns of damage.

TABLE 3
Subject Demographics and Exposure

| | | | | | | | |
|---|---|---|---|---|---|---|---|
| | | | Min Years | Age at 1st | Exposure | | |
| Sub # | Sex | Residence Zone | Exposure | Cancer Diag. | Grade | Smoker | Primary Cancer |

Exposed Subjects

| | | | | | | | |
|---|---|---|---|---|---|---|---|
| 1 | M | Red Zone | 7 | 47 | 3 | Yes | Prostate |
| 2 | F | Red Zone | 11 | 41 | 3 | No | Breast |
| 3 | F | Red Zone | 21 | 67 | 3 | Yes | Colon |
| 4 | F | Red Zone | 3 | 63 | 3 | No | Colon |
| 5 | F | Red Zone | 3 | 4 | 3 | No | Kidney (Wilms) |
| 6 | M | Red Zone | 21 | 75 | 3 | Yes | Colon |
| 7 | M | Red Zone | 21 | 63 | 3 | Yes | Colon |
| 8 | M | Red Zone | 5 | 45 | 3 | No | Brain |
| 9 | M | Red Zone | 18 | 71 | 3 | Yes | Colon |
| 10 | M | Red Zone | 18 | 69 | 3 | No | Colon |
| 11 | F | Red Zone | 22 | 52 | 3 | Yes | Uterus |
| 12 | F | Red Zone | 18 | 68 | 3 | No | Colon |
| 13 | F | Red Zone | 7 | 56 | 3 | Yes | Brain |
| 14 | M | Red Zone | 15 | 65 | 3 | Yes | Kidney |
| 15 | M | Shannon Non-Red Zone | 7 | 68 | 2-3 | Yes | Prostate |
| 16 | M | Shannon Non-Red Zone | 4 | 65 | 1-3 | Yes | Colon |
| 17 | F | Shannon Non-Red Zone | 16 | 54 | 1-3 | Yes | Stomach |
| 18 | M | Shannon Non-Red Zone | 9 | 39 | 1-3 | Yes | Kidney |
| 19 | M | Shannon Non-Red Zone | 19 | 53 | 1-3 | Yes | Kidney |
| 20 | M | Shannon Non-Red Zone | 5 | 48 | 2-3 | Yes | Kidney |
| 21 | M | Shannon Non-Red Zone | 4 | 20 | 2-3 | No | Lymphoma |
| 22 | M | Shannon Non-Red Zone | 2 | 59 | 2-3 | Yes | Esophagus |
| 23 | M | Shannon Non-Red Zone | 21 | 9 | 1-3 | No | Sarcoma |
| 24 | F | Shannon Non-Red Zone | 8 | 40 | 1-3 | No | Thyroid |
| 25 | F | Shannon Non-Red Zone | 8 | 34 | 3 | No | Uterus |
| 26 | M | Shannon Non-Red Zone | 4 | 17 | 2-3 | Yes | Brain |
| 27 | M | Shannon Non-Red Zone | 20 | 56 | 1-3 | No | Prostate |

Sporadic (Unexposed) Subjects

| | | | | | | | |
|---|---|---|---|---|---|---|---|
| 28 | F | Quebec Control | 0 | — | 0 | — | Breast |
| 29 | M | Quebec Control | 0 | — | 0 | — | Brain |
| 30 | — | Tissue Bank Control | 0 | — | 0 | — | Brain |
| 31 | — | Tissue Bank Control | 0 | — | 0 | — | Brain |
| 32 | — | Tissue Bank Control | 0 | — | 0 | — | Brain |
| 33 | — | Tissue Bank Control | 0 | — | 0 | — | Brain |
| 34 | — | Tissue Bank Control | 0 | — | 0 | — | Hemangioblastoma |
| 35 | — | Tissue Bank Control | 0 | — | 0 | — | Brain |
| 36 | F | Shannon Control | ~0 | 37 | 0-1 | Yes | Breast |
| 37 | M | Shannon Control | ~0 | 54 | 0-1 | No | Prostate |

Demographics and exposure data on subjects in the study. Years of exposure are calculated as the years a subject resided at an address with contaminated potable water after 1980 until either the resident moved or the well was sealed. Smoker indicates whether the subject was ever a regular smoker.

TABLE 4
DISTRIBUTION OF LOSS OF HETEROZYGOSITY MUTATIONS
IN EXPOSED AND SPORADIC CANCERS

| | | | | | | | | | | | | |
|---|---|---|---|---|---|---|---|---|---|---|---|---|
| | Primary | | | | | | | | | | | |
| Pt | Cancer | P26.1 | P25.3 | P24.3 | P24.2 | P24.2 | P23 | P22.1 | P12.3 | P12.1 | Q13.11 | Q13.31 |

Exposed Subjects

| | | | | | | | | | | | | |
|---|---|---|---|---|---|---|---|---|---|---|---|---|
| 1 | Prostate | NI | NO | NI | NO | NO | LOH | NO | LOH | NO | NO | LOH |
| 2 | Breast | LOH | NO | LOH | LOH | LOH | NI | LOH | LOH | LOH | LOH | LOH |
| 3 | Colon | LOH | NI | NO | NI | NO | NI | NO | LOH | NI | NO | NO |
| 4 | Colon | NI | NI | NI | LOH | NI | NI | NI | LOH | NO | NI | NO |
| 5 | Kidney | NI | LOH | NO | NI | NI | NI | NI | NI | NO | NO | NI |
| | (Wilms) | | | | | | | | | | | |
| 6 | Colon | NI | NI | NO | LOH | LOH | NI | NI | NI | LOH | NI | NO |
| 7 | Colon | NI | NI | NI | NI | NI | NI | LOH | LOH | NO | LOH | NI |
| 8 | Brain | NI | NI | NO | NI | LOH | NO | LOH | LOH | NI | NI | LOH |
| 9 | Colon | NI | NI | NO | NO | LOH | NI | NI | NI | NI | NI | NI |
| 10 | Colon | NO | LOH | NO | NO | LOH | NI | NO | LOH | NI | LOH | NO |
| 11 | Uterus | NO | NI | LOH | NI | NI | NI | NO | NO | NI | NI | NI |
| 12 | Colon | MSI | NI | MSI | NO | MSI | NO | MSI | LOH | NI | MSI | MSI |
| 13 | Brain | LOH | NO | LOH | NO | NO | LOH | NI | LOH | NO | NI | LOH |
| 14 | Kidney | NO | LOH | NI | NO | NI | NO | NI | NO | LOH | NI | LOH |
| 15 | Prostate | NO | NI | NO | NI | NI | NI | NI | LOH | NI | NI | NI |
| 16 | Colon | LOH | NI | NI | NI | NO | NI | NI | LOH | NO | LOH | LOH |
| 17 | Stomach | NO | NI | NI | Nl | NI | NO | NI | NI | NO | LOH | LOH |
| 18 | Kidney | NI | LOH | LOH | NI | LOH | NI | NI | LOH | NI | NI | NO |
| 19 | Kidney | NI | NO | NI | NO | NI | NO | NI | NI | NO | NI | NO |
| 20 | Kidney | NI | LOH | NO | NI | NO | NI | NI | NI | LOH | NI | NO |
| 21 | Lymphoma | LOH | NO | LOH | NO | NI | NI | NO | NO | LOH | NI | NO |
| 22 | Esophagus | NO | LOH | LOH | NI | NI | NO | LOH | NI | NI | NI | NO |
| 23 | Sarcoma | LOH | NO | NO | NI | NI | NO | LOH | NI | NI | NI | NO |
| 24 | Thyroid | NI | NO | LOH | NI | Nl | NI | NO | LOH | LOH | Nl | NI |
| 25 | Uterus | NO | NI | NI | NI | NI | NI | NO | NI | LOH | NO | NO |
| 26 | Brain | NO | NO | Nl | NO | NO | NI | NO | LOH | NO | NI | LOH |
| 27 | Prostate | NO | NI | NI | NI | NI | NI | NI | LOH | LOH | LOH | NI |

Sporadic (Unexposed) Subjects

| | | | | | | | | | | | | |
|---|---|---|---|---|---|---|---|---|---|---|---|---|
| 28 | Breast | NO | NI | NO | NO | NI | NO | NO | NI | NO | Nl | NO |
| 29 | Brain | NO | NI | NI | NO | NO | NO | NI | LOH | NO | LOH | NO |
| 30 | Brain | NI | NO | NI | NI | NO | NO | NO | NO | NO | LOH | NO |
| 31 | Brain | NO | NO | NO | NO | NO | NO | NI | NO | NI | NI | NO |
| 32 | Brain | NI | NI | NO | NI | NI | NO | NO | NO | NI | NO | NO |
| 33 | Brain | LOH | NO | NO | NI | NO | NI | NI | NO | NI | NO | NO |
| 34 | HB | NI | NI | NO | NO | NO | NO | NO | NO | NO | NI | NI |
| 35 | Brain | NI | NO | NI | NI | NO | NO | NO | NO | NI | NO | NI |
| 36 | Breast | NI | NO | NO | Nl | NI | NO | NI | NO | NI | NI | NO |
| 37 | Prostate | NO | NO | NI | NO | NO | NO | NI | LOH | NO | NO | NO |

| | Primary | | | | | | |
|---|---|---|---|---|---|---|---|
| Pt | Cancer | Q21.3 | Q22.1 | Q26.1 | Q26.31 | Q26.31 | FAL |
| Exposed Subjects | | | | | | | |
| 1 | Prostate | NO | LOH | NO | NI | LOH | 5/13 = 0.38 |
| 2 | Breast | LOH | NI | LOH | LOH | LOH | 13/14 = 0.93 |
| 3 | Colon | NO | LOH | NO | LOH | NO | 4/12 = 0.33 |
| 4 | Colon | LOH | NI | NO | LOH | LOH | 5/8 = 0.63 |
| 5 | Kidney | NI | LOH | NI | NO | LOH | 3/7 = 0.43 |
| | (Wilms) | | | | | | |
| 6 | Colon | NI | LOH | NO | NI | NI | 4/7 = 0.57 |
| 7 | Colon | NI | LOH | LOH | NI | NO | 5/7 = 0.71 |
| 8 | Brain | NI | NO | LOH | NI | NO | 5/9 = 0.56 |
| 9 | Colon | LOH | LOH | NO | LOH | NI | 4/7 = 0.57 |
| 10 | Colon | NI | NO | NI | NI | NI | 4/10 = 0.4 |
| 11 | Uterus | LOH | LOH | NO | NO | LOH | 4/9 = 0.44 |
| 12 | Colon | MSI | MSI | MSI | MSI | NI | — |
| 13 | Brain | NI | NO | LOH | LOH | NO | 7/13 = 0.54 |
| 14 | Kidney | NI | LOH | NO | LOH | NI | 5/10 = 0.5 |
| 15 | Prostate | LOH | NI | NO | NO | NI | 2/6 = 0.33 |
| 16 | Colon | NI | NI | NO | LOH | NI | 5/8 = 0.63 |
| 17 | Stomach | NI | NO | NI | NI | NO | 2/7 = 0.29 |
| 18 | Kidney | NI | NI | LOH | NI | NI | 5/6 = 0.83 |
| 19 | Kidney | NO | NI | NO | NI | NO | 0/8 = 0 |
| 20 | Kidney | Nl | LOH | NO | LOH | NI | 4/8 = 0.5 |
| 21 | Lymphoma | NO | NO | LOH | NI | NO | 4/12 = 0.33 |
| 22 | Esophagus | LOH | NI | NI | NI | LOH | 5/8 = 0.63 |
| 23 | Sarcoma | LOH | LOH | LOH | NI | NI | 5/9 = 0.56 |
| 24 | Thyroid | NO | LOH | NO | NO | LOH | 5/10 = 0.5 |
| 25 | Uterus | LOH | NO | NI | LOH | NI | 3/8 = 0.38 |
| 26 | Brain | NO | LOH | NO | NI | NI | 3/11 = 0.27 |
| 27 | Prostate | LOH | LOH | NI | NO | NI | 5/7 = 0.71 |
| Sporadic (Unexposed) Subjects | | | | | | | |
| 28 | Breast | LOH | NO | NI | NO | NI | 1/10 = 0.1 |
| 29 | Brain | NI | NO | NO | NO | NI | 2/11 = 0.18 |
| 30 | Brain | NI | NO | NO | NI | NI | 1/10 = 0.1 |
| 31 | Brain | NI | NO | LOH | NI | NI | 1/10 = 0.1 |
| 32 | Brain | NI | LOH | NI | NI | NI | 1/7 = 0.14 |
| 33 | Brain | NO | NO | NI | NI | NI | 1/9 = 0.11 |
| 34 | HB | NI | LOH | NI | NO | NI | 1/9 = 0.11 |
| 35 | Brain | NI | NI | NI | NO | NI | 0/7 = 0 |
| 36 | Breast | NI | NO | NI | NO | NO | 0/8 = 0 |
| 37 | Prostate | NI | NO | NO | NI | NO | 1/12 = 0.08 |

Abbreviations.
NI = noninformative status for the particular microsatellite marker,
NO = no LOH detected,
LOH = loss of heterozygosity detected (see methods for details),
MSI = microsatellite instability present precluding LOH analysis.
HB = Hemangioblastoma,
GBM = Glioblastoma multiforme,
LG ASTRO = low grade astrocytoma.

TABLE 5

| | | FAL | | | | | |
|---|---|---|---|---|---|---|---|
| | Number | (Fractional | | | TOTAL | | |
| | of | Allelic Loss) | | | MUTATIONS | | |
| Exposure | Subjects | Avg. | Std. | Range | Avg. | Std. | Range |
| Exposed | 26 | 0.50 | 0.20 | 0.0-0.9 | 4.5 | 2.2 | 0-13 |
| Unexposed | 10 | 0.09 | 0.06 | 0.0-0.18 | 0.9 | 0.6 | 0-2 |

FAL represents fractional allelic loss rate (see methods for description). Total mutations represents the detectable mutations for each cancer irrespective of the number of informative markers.

Although the present invention has been described in considerable detail with reference to certain preferred embodiments thereof, other versions are possible. Therefore the spirit and scope of the appended claims should not be limited to the description and the preferred versions contained within this specification.

In the present disclosure, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figure, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” at least and the like include the number recited and refer to ranges which can be subsequently broken down into subranges as discussed above. Finally, as will be understood by one skilled in the art, a range includes each individual member.

What is claimed is:

1. A method of differentiating a non-genotoxin-associated tumor from a genotoxin-associated tumor comprising:
counting the number of loci from a set of loci spanning a segment of a genome not containing a tumor suppressor gene with copy number imbalance, wherein a tumor is a genotoxin-associated tumor when about 20% or more of the loci show copy number imbalance.

2. The method of claim 1 further comprising determining the presence of a point mutation in a VHL gene, wherein a point mutation in distal portion of exon 1 of the VHL gene is indicative of a genotoxin-associated tumor.

3. The method of claim 2, wherein the distal portion of exon 1 comprises codon 81.

4. The method of claim 1 further comprising determining the presence of point mutation in exons 1, 2 and 3 of the VHL gene, wherein the presence of more than one point mutation in said exons is indicative of a genotoxin-associated tumor.

5. The method of claim 1, wherein said genotoxin comprises a carcinogen, a chlorinated hydrocarbon, a polycyclic aromatic hydrocarbon, a benzene, aflatoxin, or a combination thereof.

6. The method of claim 5, wherein the chlorinated hydrocarbon comprises trichloroethylene, vinyl chloride or a combination thereof.

7. The method of claim 1, wherein a non-genotoxin associated tumor comprises a sporadic tumor.

8. The method of claim 1, wherein the genotoxin-associated tumor is from a subject who has a cancer.

9. The method of claim 7, wherein said cancer is selected from colon, brain, breast, kidney, leukemia, prostate, uterus, stomach, lymphoma, esophagus, sarcoma, thyroid, hemangioblastoma and combinations thereof.

10. The method of claim 1 further comprising determining passenger fractional allelic loss mutation rate in a segment of a genome wherein a fractional allelic loss mutation rate of about 20% or more is indicative of a genotoxin-associated tumor.

11. The method of claim 1, wherein the segment of a genome comprises chromosome 3, chromosome 4, chromosome 12, chromosome 16 or a combination thereof.

12. The method of claim 1, wherein the segment of a genome does not contain a tumor suppressor gene.
